(19) United States
(12) Patent Application Publication
(10) Pub. No.: US 2004/0248897 A1
(43) Pub. Date: Dec. 9, 2004

(54) Substituted N-acylaniline derivatives, the preparation thereof, and their use as pharmaceutical compositions

(75) Inventors: Henning Priepke, Warthausen (DE); Norbert Hauel, Schemmerhofen (DE); Armin Heckel, Biberach (DE); Uwe Ries, Biberach (DE); Klaus Binder, Blaustein (DE); Rainer Zimmermann, Mittlebiberach (DE); Jean-Marie Stassen, Lubbeek (BE); Wolfgang Wienen, Biberach (DE)

Correspondence Address: BOEHRINGER INGELHEIM CORPORATION, 900 RIDGEBURY ROAD, P. O. BOX 368, RIDGEFIELD, CT, 06877, US

(73) Assignee: Boehringer Ingelheim Pharma GmbH & Co. KG, Ingelheim (DE)

(21) Appl. No.: 10/743,159

(22) Filed: Dec. 22, 2003

Related U.S. Application Data
(63) Continuation of application No. PCT/EP02/06774, filed on Jun. 19, 2002.

(30) Foreign Application Priority Data
Jun. 23, 2001 (DE) 101 30 374.2

(51) Int. Cl. A61K 31/55; A61K 31/53; A61K 31/501; A61K 31/497; A61K 31/4965

(52) U.S. Cl. 514/242; 514/247; 514/252.1; 514/256; 514/357; 514/617; 514/217.05; 514/217.06; 514/255.05; 514/252.03; 514/252.05; 514/318

(57) ABSTRACT

Substituted N-acylaniline derivatives of general formula

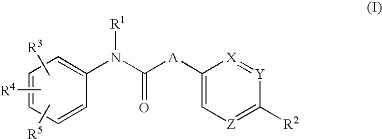

wherein R¹ to R⁵, A, X, Y, and Z are as defined herein, and the tautomers, prodrugs, and derivatives thereof which contain, instead of a carboxy group, a group which is negatively charged under physiological conditions contain, and the salts thereof, particularly the physiologically acceptable salts thereof with inorganic or organic acids or bases, which have valuable properties.

### RELATED APPLICATIONS

This application is a continuation under 35 U.S.C. § 365(c) of International Application No. PCT/EP02/06774, filed Jun. 19, 2002, which claimed priority to German Application No. 101 30 374.2, filed Jun. 23, 2001, each of which is hereby incorporated by reference in its entirety.

### FIELD OF THE INVENTION

The present invention relates to new substituted N-acylaniline derivatives of general formula

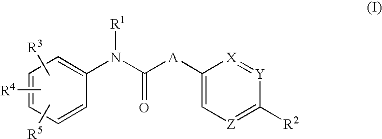

the tautomers, the stereoisomers, the prodrugs thereof, the mixtures thereof, and the salts thereof, particularly the physiologically acceptable salts thereof with inorganic or organic acids or bases, which have valuable properties.

The compounds of the above general formula I wherein R² does not contain a cyano group have valuable pharmacological properties, particularly thrombin-inhibiting properties, and

the compounds of the above general formula I wherein R² contains a cyano group constitute valuable intermediate products for preparing the compounds of general formula I wherein R² denotes an optionally substituted amidino or aminomethyl group.

The present invention thus relates to the new compounds of the above general formula I as well as the preparation thereof, the pharmaceutical compositions containing the pharmacologically effective compounds and the use thereof.

In the above general formula

A denotes a methylene group optionally substituted by a C₁₋₃-alkyl group, or a straight-chain C₂₋₃-alkyl group optionally substituted by a C₁₋₃-alkyl group wherein the methylene group linked to the aromatic group or heteroaromatic group may be replaced by an oxygen or sulfur atom or by an —NH— group, while the —NH— group may additionally be substituted by a C₁₋₃-alkyl, carboxy-C₁₋₃-alkyl or C₁₋₃-alkoxycarbonyl-C₁₋₃-alkyl group,

R¹ denotes a hydrogen atom or a C₁₋₃-alkyl group optionally substituted by a carboxy group,

R² denotes a cyano, aminomethyl or amidino group,

R³ denotes a C₁₋₅-alkyl or carboxy-C₁₋₄-alkyl group which may in each case be substituted in the alkyl moiety by a C₃₋₇-cycloalkyl, phenyl, pyridyl, pyrrolidino, 2,5-dihydro-1H-pyrrolino, piperidino or hexamethyleneimino group,

a carbonyl or sulfonyl group which is substituted in each case

by a C₁₋₅-alkyl, C₃₋₇-cycloalkyl or phenyl group optionally substituted by a C₁₋₃-alkyl or carboxy-C₁₋₃-alkyl group,

by an amino, C₁₋₄-alkylamino or carboxy-C₁₋₄-alkylamino group substituted by a C₁₋₅-alkyl, C₃₋₇-cycloalkyl, phenyl, phenyl-C₁₋₃-alkyl, pyridyl or pyridyl-C₁₋₃-alkyl group or

by a pyrrolidino, 2,5-dihydro-1H-pyrrolino, piperidino or hexamethyleneimino group optionally substituted by a C₁₋₃-alkyl or carboxy-C₁₋₃-alkyl group,

a carboxy-C₁₋₃-alkylcarbonylamino group optionally substituted in the alkyl moiety by an amino, C₁₋₃-alkylamino or di-(C₁₋₃-alkyl)-amino group, an amino, carboxy-C₁₋₃-alkylaminocarbonylamino, carboxy-C₁₋₃-alkylaminocarbonyl-C₁₋₃-alkylcarbonylamino, carboxy-C₁₋₃-alkylaminocarbonyl-C₁₋₃-alkylaminocarbonylamino, amino-C₁₋₃-alkylcarbonylamino, C₁₋₃-alkylamino-C₁₋₃-alkylcarbonylamino or di-(C₁₋₃-alkyl)-amino-C₁₋₃-alkylcarbonylamino group, while in each case in the abovementioned amino groups

the hydrogen atom of the amino group which is linked to the phenyl ring is replaced by a C₁₋₆-alkyl, C₃₋₇-cycloalkyl, phenyl or pyridyl group, an n-propylene or n-butylene bridge or a phenyl, pyridine or piperidine ring may each be fused to the abovementioned phenyl or pyridyl substituents via two adjacent carbon atoms, or the abovementioned aromatic substituents may each additionally be substituted by a C₁₋₃-alkyl, C₁₋₃-alkyloxy, trifluoromethyl or carboxy group or by 2 to 4 methyl groups,

an amino, carboxy-C₁₋₄-alkylamino, carboxy-C₁₋₃-alkylaminocarbonyl-C₁₋₃-alkylamino, aminocarbonyl-C₁₋₃-alkylamino, C₁₋₃-alkylaminocarbonyl-C₁₋₃-alkylamino, di-(C₁₋₃-alkyl)-aminocarbonyl-C₁₋₃-alkylamino, amino-C₂₋₃-alkylamino, C₁₋₄-alkylamino-C₂₋₃-alkylamino, di-(C₁₋₄-alkyl)-amino-C₂₋₃-alkylamino, pyrrolidinocarbonyl-C₁₋₃-alkylamino, piperidinocarbonyl-C₁₋₃-alkylamino, hexahydroazepinocarbonyl-C₁₋₃-alkylamino, morpholinocarbonyl-C₁₋₃-alkylamino, piperazinocarbonyl-C₁₋₃-alkylamino or N-(C₁₋₃-alkyl)-piperazinocarbonyl-C₁₋₃-alkylamino group, while in each case in the abovementioned amino groups

the hydrogen atom of the amino group which is linked to the phenyl ring is replaced by a C₁₋₅-alkylcarbonyl, C₁₋₅-alkylsulfonyl, C₃₋₇-cycloalkylcarbonyl, C₃₋₇-cycloalkylsulfonyl, benzoyl, phenylsulfonyl, phenyl-C₁₋₃-alkylcarbonyl, phenyl-C₁₋₃-alkylsulfonyl or pyridinoyl group, an n-propylene or n-butylene bridge or a phenyl, pyridine or piperidine ring may each be fused to the abovementioned phenyl or pyridyl substituents via two adjacent carbon atoms or the abovementioned aromatic substituents may each additionally be substituted by a C₁₋₃-alkyl, C₁₋₃-alkyloxy, trifluoromethyl or carboxy group or by 2 to 4 methyl groups,

a phenyl, pyridyl, imidazolyl or pyrazolyl group optionally substituted by one, two or three C₁₋₃-alkyl groups, while in each case the alkyl substituents may be identical or different and one of the alkyl substituents may additionally be substituted by a carboxy, hydroxysulfonyl, aminosulfonyl, C₁₋₄-alkylaminosulfonyl, di-(C₁₋₄-alkyl)-aminosulfonyl or C₁₋₄-alkylsulfonyl group,

R⁴ denotes a fluorine, chlorine, bromine or iodine atom, a carboxy, C₁₋₃-alkyl, carboxy-C₁₋₃-alkyl, trifluoromethyl or C₁₋₃-alkoxy group or also a hydrogen atom, if

R³ denotes a C₁₋₅-alkyl or carboxy-C₁₋₄-alkyl group which is substituted in each case in the alkyl moiety by a C₃₋₇-cycloalkyl, phenyl, pyridyl, pyrrolidino, 2,5-dihydro-1H-pyrrolino, piperidino or hexamethyleneimino group,

an amino, carboxy-C₁₋₄-alkylamino or carboxy-C₁₋₃-alkylaminocarbonyl-C₁₋₃-alkylamino group, while in each case in the abovementioned amino groups

the hydrogen atom of the amino group which is linked to the phenyl ring is replaced by a C₁₋₅-alkylcarbonyl, C₁₋₅-alkylsulfonyl, C₃₋₇-cycloalkylcarbonyl, C₃₋₇-cycloalkylsulfonyl, benzoyl, phenylsulfonyl, phenyl-C₁₋₃-alkylcarbonyl, phenyl-C₁₋₃-alkylsulfonyl or pyridinoyl group, an n-propylene or n-butylene bridge, a phenyl, pyridine or piperidine ring may each be fused to the abovementioned phenyl or pyridyl substituents via two adjacent carbon atoms or the abovementioned aromatic substituents may each additionally be substituted by a C₁₋₃-alkyl, C₁₋₃-alkyloxy, trifluoromethyl or carboxy group or by 2 to 4 methyl groups,

R⁵ denotes a hydrogen, fluorine, chlorine, bromine or iodine atom, a C₁₋₃-alkyl or trifluoromethyl group or

R⁴ and R⁵ together denote an n-C₃₋₄-alkylene group,

with the proviso that at least one of the groups R¹, R⁴ or R⁵ is not a hydrogen atom, and

X, Y, and Z in each case represent nitrogen atoms or —CH— groups, with the proviso that at least one of the groups X, Y, and Z denotes a —CH— group,

while, unless otherwise stated, the hydrogen atoms in the methyl and methoxy groups mentioned in the definition of the above groups or in the methyl moieties contained in the above-defined groups of formula I may be wholly or partially replaced by fluorine atoms, and

the carboxy groups mentioned in the definition of the above groups may be replaced by a group which may be converted in vivo into a carboxy group or by a group which is negatively charged under physiological conditions or

the amino and imino groups mentioned in the definition of the above groups may be substituted by a group which can be cleaved in vivo.

“Prodrug groups” of this kind are described, for example, in WO 98/46576 and by N. M. Nielson et al., International Journal of Pharmaceutics 39, 75-85 (1987).

For example, the carboxy groups mentioned above in the definition of the groups may be replaced by a tetrazolyl group or by a group which may be converted in vivo into a carboxy group, e.g., by a hydroxymethyl or formyl group, by a carboxy group esterified with an alcohol wherein the alcohol moiety is preferably a C₁₋₆-alkanol, a phenyl-C₁₋₃-alkanol, a C₃₋₉-cycloalkanol, while a C₅₋₈-cycloalkanol may additionally be substituted by one or two C₁₋₃-alkyl groups, a C₅₋₈-cycloalkanol wherein a methylene group in the 3 or 4 position is replaced by an oxygen atom or by an imino group optionally substituted by a C₁₋₃-alkyl, phenyl-C₁₋₃-alkyl, phenyl-C₁₋₃-alkoxycarbonyl or C₂₋₆-alkoxycarbonyl or C₂₋₆-alkanoyl group and the cycloalkanol moiety may additionally be substituted by one or two C₁₋₃-alkyl groups, a C₄₋₇-cycloalkenol, a C₃₋₅-alkenol, a phenyl-C₃₋₅-alkenol, a C₃₋₅-alkynol or phenyl-C₃₋₅-alkynol, with the proviso that no bond to the oxygen atom starts from a carbon atom which carries a double or triple bond, a C₃₋₈-cycloalkyl-C₁₋₃-alkanol, a bicycloalkanol with a total of 8 to 10 carbon atoms which is additionally substituted in the bicycloalkyl moiety by one or two C₁₋₃-alkyl groups, a 1,3-dihydro-oxo-1-isobenzofuranol or an alcohol of formula

RₐCO—O—(R_{b}CR_{c})—OH, wherein

Rₐ denotes a C₁₋₈-alkyl, C₅₋₇-cycloalkyl, phenyl or phenyl-C₁₋₃-alkyl group,

R_{b} denotes a hydrogen atom, a C₁₋₃-alkyl, C₅₋₇-cycloalkyl or phenyl group, and

R_{c} denotes a hydrogen atom or a C₁₋₃-alkyl group,

by a group which is negatively charged under physiological conditions such as a tetrazol-5-yl, phenylcarbonylaminocarbonyl, trifluoromethylcarbonylaminocarbonyl, C₁₋₆-alkylsulfonylamino, phenylsulfonylamino, benzylsulfonylamino, trifluoromethyl-sulfonylamino, C₁₋₄-alkylsulfonylaminocarbonyl, phenylsulfonylaminocarbonyl, benzylsulfonylaminocarbonyl or perfluoro-C₁₋₆-alkylsulfonylaminocarbonyl group

and the imino or amino groups mentioned in the definition of the groups may be substituted by a group which can be cleaved in vivo, e.g., by a hydroxy-C₁₋₈-alkoxy, allyloxy, phenyloxy, benzyloxy, 3-methoxybenzyloxy, 4-methylbenzyloxy or 4-chlorophenyl-C₁₋₆-alkyloxy group, by an acyl group such as the benzoyl or pyridinoyl group or a C₁₋₁₆-alkanoyl group such as the formyl, acetyl, propionyl, butanoyl, pentanoyl or hexanoyl group, by an allyloxycarbonyl group, by a C₁₋₁₆-alkoxycarbonyl group such as the methyloxycarbonyl, ethyloxycarbonyl, propyloxycarbonyl, isopropyloxycarbonyl, butyloxycarbonyl, tert-butyloxycarbonyl, pentyloxycarbonyl, hexyloxycarbonyl, octyloxycarbonyl, nonyloxycarbonyl, decyloxycarbonyl, undecyloxycarbonyl, dodecyloxycarbonyl or hexadecyloxycarbonyl group, by a phenyl-C₁₋₁₆-alkoxycarbonyl group such as the benzyloxycarbonyl, phenylethyloxycarbonyl or phenylpropyloxycarbonyl group, by a C₁₋₃-alkylsulfonyl-C₂₋₄-alkoxycarbonyl, C₁₋₃-alkoxy-C₂₋₄-alkoxy-C₂₋₄-alkoxycarbonyl or RₐCO—O—(R_{b}CR_{c})—O—CO group wherein Rₐ to R_{b} are as hereinbefore defined.

Moreover, the saturated alkyl and alkoxy moieties which contain more than 2 carbon atoms, as well as the alkanoyl and unsaturated alkyl moieties which contain more than 3 carbon atoms mentioned in the above definitions also include the branched isomers thereof, such as for example the isopropyl, tert-butyl, isobutyl group, etc.

Preferred compounds of general formula I are those wherein

A denotes a methylene group or

a C₂₋₃-alkyl group wherein the methylene group linked to the aromatic group or heteroaromatic group may be replaced by an —NH— group or by an oxygen atom, while the —NH— group may additionally be substituted by a C₁₋₃-alkyl, carboxy-C₁₋₃-alkyl or C₁₋₃-alkoxycarbonyl-C₁₋₃-alkyl group,

R¹ denotes a hydrogen atom or a C₁₋₃-alkyl group optionally substituted by a carboxy group,

R² denotes a cyano or aminomethyl group or an amidino group optionally substituted by a hydroxy, C₁₋₈-alkoxycarbonyl or benzoyl group,

R³ denotes a C₁₋₅-alkyl or carboxy-C₁₋₄-alkyl group which may be substituted in the alkyl moiety in each case by a C₃₋₇-cycloalkyl, phenyl, pyridyl, pyrrolidino, 2,5-dihydro-1H-pyrrolino, piperidino or hexamethyleneimino group,

a carbonyl or sulfonyl group which is substituted in each case

by a C₁₋₅-alkyl or C₃₋₇-cycloalkyl group optionally substituted by a C₁₋₃-alkyl or carboxy-C₁₋₃-alkyl group,

by an amino, C₁₋₄-alkylamino or carboxy-C₁₋₄-alkylamino group substituted by a C₁₋₅-alkyl, C₃₋₇-cycloalkyl, phenyl, benzyl or pyridyl group or

by a pyrrolidino, 2,5-dihydro-1H-pyrrolino, piperidino or hexamethyleneimino group optionally substituted by a C₁₋₃-alkyl or carboxy-C₁₋₃-alkyl group,

a carboxy-C₁₋₃-alkylcarbonylamino group optionally substituted in the alkyl moiety by an amino group, an amino, carboxy-C₁₋₃-alkylaminocarbonylamino, carboxy-C₁₋₃-alkylaminocarbonyl-C₁₋₃-alkylcarbonylamino, carboxy-C₁₋₃-alkylaminocarbonyl-C₁₋₃-alkylaminocarbonylamino or amino-C₁₋₃-alkylcarbonylamino group, while in each case in the abovementioned amino groups

the hydrogen atom of the amino group which is linked to the phenyl ring is replaced by a C₁₋₆-alkyl, C₃₋₇-cycloalkyl, phenyl or pyridyl group, a phenyl, pyridine or piperidine ring may be fused to the abovementioned phenyl or pyridyl substituents in each case via two adjacent carbon atoms or the abovementioned aromatic substituents may each additionally be substituted by a C₁₋₃-alkyl, C₁₋₃-alkyloxy or trifluoromethyl group or by 2 to 4 methyl groups,

an amino, carboxy-C₁₋₄-alkylamino, carboxy-C₁₋₃-alkylaminocarbonyl-C₁₋₃-alkylamino, aminocarbonyl-C₁₋₃-alkylamino, pyrrolidinocarbonyl-C₁₋₃-alkylamino, piperidinocarbonyl-C₁₋₃-alkylamino or morpholinocarbonyl-C₁₋₃-alkylamino group, while in each case in the abovementioned amino groups

the hydrogen atom of the amino group which is linked to the phenyl ring is replaced by a C₁₋₅-alkylcarbonyl, C₁₋₅-alkylsulfonyl, C₃₋₇-cycloalkylcarbonyl, C₃₋₇-cycloalkylsulfonyl, benzoyl, phenylsulfonyl, phenyl-C₁₋₃-alkylcarbonyl or pyridinoyl group, an n-propylene or n-butylene bridge or a phenyl, pyridine or piperidine ring may each be fused to the abovementioned phenyl or pyridyl substituents via two adjacent carbon atoms or the abovementioned aromatic substituents may each additionally be substituted by a C₁₋₃-alkyl, C₁₋₃-alkyloxy, trifluoromethyl or carboxy group or by 2 to 4 methyl groups,

a phenyl, pyridyl, imidazolyl or pyrazolyl group optionally substituted by one, two or three C₁₋₃-alkyl groups, while in each case the alkyl substituents may be identical or different and one of the alkyl substituents may additionally be substituted by a carboxy or hydroxysulfonyl group, an aminosulfonyl, C₁₋₄-alkylaminosulfonyl, di-(C₁₋₄-alkyl)-aminosulfonyl or C₁₋₄-alkylsulfonyl group,

R⁴ denotes a chlorine or bromine atom, a carboxy, C₁₋₃-alkyl, carboxy-C₁₋₃-alkyl or trifluoromethyl group or also a hydrogen atom, if

R³ denotes a C₁₋₅-alkyl or carboxy-C₁₋₄-alkyl group which is substituted in each case in the alkyl moiety by a C₃₋₇-cycloalkyl, phenyl, pyridyl, pyrrolidino, piperidino or hexamethyleneimino group,

an amino, carboxy-C₁₋₄-alkylamino or carboxy-C₁₋₃-alkylaminocarbonyl-C₁₋₃-alkylamino group, while in each case in the abovementioned amino groups

the hydrogen atom of the amino group which is linked to the phenyl ring is replaced by a C₁₋₅-alkylcarbonyl, C₁₋₅-alkylsulfonyl, C₃₋₇-cycloalkylcarbonyl, C₃₋₇-cycloalkylsulfonyl, benzoyl, phenylsulfonyl, phenyl-C₁₋₃-alkylcarbonyl or pyridinoyl group, an n-propylene or n-butylene bridge or a phenyl, pyridine or piperidine ring may each be fused to the abovementioned phenyl or pyridyl substituents via two adjacent carbon atoms or the abovementioned aromatic substituents may each additionally be substituted by a C₁₋₃-alkyl, C₁₋₃-alkyloxy or trifluoromethyl group or by 2 to 4 methyl groups,

R⁵ denotes a hydrogen, chlorine or bromine atom, a C₁₋₃-alkyl or trifluoromethyl group or

R⁴ and R⁵ together represent an n-C₃₋₄-alkylene group,

with the proviso that at least one of the groups R¹, R⁴ or R⁵ is not a hydrogen atom, and

X, Y, and Z in each case represent nitrogen atoms or —CH— groups, with the proviso that at least one of the groups X, Y, and Z denotes a —CH— group,

the C₁₋₃-alkyl and benzyl esters thereof, the tautomers, the stereoisomers, the prodrugs thereof, the mixtures thereof, and the salts thereof,

while, unless otherwise stated, the hydrogen atoms in the methyl and methoxy groups mentioned in the definition of the above groups or in the methyl moieties contained in the above-defined groups of formula I may be wholly or partially replaced by fluorine atoms, and

the saturated alkyl and alkoxy moieties which contain more than 2 carbon atoms, as well as the alkanoyl and unsaturated alkyl moieties which contain more than 3 carbon atoms mentioned in the above definitions also include the branched isomers thereof, such as for example the isopropyl, tert-butyl, isobutyl group, etc.

Particularly preferred compounds of the present invention are the compounds of general formula Ia

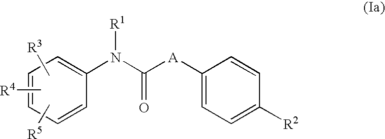

wherein

A denotes a methylene group or

an ethylene group wherein the methylene group linked to the aromatic group may be replaced by an oxygen atom or by an —NH— group, while the —NH— group may additionally be substituted by a methyl, carboxymethyl or C₁₋₃-alkoxycarbonylmethyl group,

R¹ denotes a hydrogen atom, a methyl or ethyl group,

R² denotes a cyano or aminomethyl group or an amidino group optionally substituted by a hydroxy, C₁₋₈-alkyloxycarbonyl or benzoyl group,

R³ denotes a straight-chain or branched C₁₋₅-alkyl group optionally substituted by a phenyl, pyridyl or piperidino group,

a carbonyl or sulfonyl group which is substituted in each case by a straight-chain or branched C₁₋₅-alkyl, C₃₋₅-cycloalkyl, phenylamino, N-(C₁₋₄-alkyl)-phenylamino, N,N-di-(C₁₋₄-alkyl)-amino, N-(C₁₋₄-alkyl)-benzylamino, N-(C₁₋₄-alkyl)-pyridylamino, pyrrolidino or methylpyrrolidino group,

an amino, methylamino, carboxymethylamino, C₁₋₃-alkoxycarbonylmethylamino or morpholinocarbonylmethylamino group which is substituted in each case at the amino nitrogen atom by a phenylsulfonyl group optionally substituted by one to four methyl groups, by a phenylsulfonyl group substituted by a trifluoromethyl, carboxy or C₁₋₃-alkoxycarbonyl group, by

a benzoyl, benzylsulfonyl, naphthylsulfonyl, quinolylsulfonyl or 1,2,3,4-tetrahydroquinolylsulfonyl group, or

a straight-chain or branched C₁₋₅-alkylamino or C₃₋₅-cycloalkylamino group which is substituted in each case at the amino nitrogen atom by a C₂₋₃-alkanoyl group substituted by a carboxy or C₁₋₃-alkoxycarbonyl and/or an amino group, by a carboxymethylaminocarbonyl or C₁₋₃-alkoxycarbonylmethylaminocarbonyl group, or

a pyrazol-1-yl group substituted by two straight-chain or branched C₁₋₃-alkyl groups,

R⁴ denotes a chlorine or bromine atom, a methyl, trifluoromethyl, carboxymethyl or C₁₋₃-alkoxycarbonylmethyl group or also a hydrogen atom, if

R¹ denotes an ethyl group or

R³ denotes a pyrrolidinocarbonyl group, a carboxymethylamino or C₁₋₃-alkoxycarbonylmethylamino group wherein in each case the amino nitrogen atom is substituted by a benzoyl group,

R⁵ denotes a hydrogen, chlorine or bromine atom or a methyl group or

R⁴ and R⁵ together denote an n-propylene group,

with the proviso that at least two of the groups R¹, R⁴, and R⁵ are not hydrogen atoms,

particularly those compounds of general formula Ia, wherein

R⁴ denotes a chlorine or bromine atom, a methyl or trifluoromethyl group,

while, unless otherwise stated, the hydrogen atoms in the methyl and methoxy groups mentioned in the definition of the above groups or in the methyl moieties contained in the above-defined groups of formula Ia may be wholly or partially replaced by fluorine atoms,

the tautomers, the stereoisomers, the prodrugs thereof, and the salts thereof.

Most particularly preferred compounds of the above general formula Ia are those wherein

A denotes an ethylene group wherein the methylene group linked to the aromatic group may be replaced by an —NH— group,

R¹ denotes a hydrogen atom, a methyl or ethyl group,

R² denotes a amidino group,

R³ denotes a C₃₋₅-alkyl group,

a carbonyl group which is substituted by a straight-chain or branched C₁₋₅-alkyl, C₃₋₅-cycloalkyl, N,N-di-(C₁₋₄-alkyl)-amino, N-(C₁₋₄-alkyl)-benzylamino, N-(C₁₋₄-alkyl)-pyridylamino, pyrrolidino or 2-methylpyrrolidino group,

a straight-chain or branched C₁₋₅-alkylamino or C₃₋₅-cycloalkylamino group which is substituted in each case at the amino nitrogen atom by a C₂₋₃-alkanoyl group substituted by a carboxy or C₁₋₃-alkoxycarbonyl and/or an amino group, by a carboxymethylaminocarbonyl or C₁₋₃-alkoxycarbonylmethylaminocarbonyl group, or

a pyrazol-1-yl group substituted by two straight-chain or branched C₁₋₃-alkyl groups,

R⁴ denotes a chlorine or bromine atom, a methyl or trifluoromethyl group, and

R⁵ denotes a hydrogen, chlorine, or bromine atom or a methyl group,

particularly those compounds of general formula Ia wherein

R³ is in the 4 position,

with the proviso that at least one of the groups R¹ or R⁵ is not a hydrogen atom,

while, unless otherwise stated, the hydrogen atoms in the methyl and methoxy groups mentioned in the definition of the above groups or in the methyl moieties contained in the above-defined groups of formula I may be wholly or partially replaced by fluorine atoms,

the tautomers, the stereoisomers, the prodrugs thereof, and the salts thereof.

The following particularly preferred compounds of general formula I may be mentioned by way of example:

(A) 4-{N-[2,5-dimethyl-4-(2-methylpyrrolidinocarbonyl)phenylaminocarbonylmethyl]-amino}benzamidine,

(B) 4-[N-(2,5-dimethyl-4-isopropylcarbonylphenylaminocarbonylmethyl)amino]benzamidine, and

(C) 4-{N-[2,5-dimethyl-4-(N′-isopropyl-N′-(2-ethoxycarbonylethylcarbonyl)amino)phenylaminocarbonylmethyl]amino}benzamidine

and the salts thereof

According to the invention the compounds of general formula I are obtained by methods known per se, for example by the following methods:

a) reacting a compound of general formula

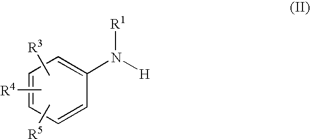

wherein

R¹ and R³ to R⁵ are as hereinbefore defined,

with a carboxylic acid of general formula

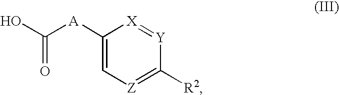

wherein

A, X, Y, Z, and R² are as hereinbefore defined, or the reactive derivatives thereof.

The reaction is preferably carried out in a solvent such as methanol, ethanol, methylene chloride, tetrahydrofuran, toluene, dioxane, dimethylsulfoxide or dimethylformamide, optionally in the presence of an inorganic or a tertiary organic base, preferably at temperatures between 20° C. and the boiling temperature of the solvent used.

The reaction is, however, preferably carried out with a carboxylic acid of general formula III in the presence of a dehydrating or acid-activating compound, e.g., in the presence of isobutyl chloroformate, thionyl chloride, trimethylchlorosilane, hydrochloric acid, sulfuric acid, methanesulfonic acid, p-toluenesulfonic acid, phosphorus trichloride, phosphorus pentoxide, N,N′-dicyclohexylcarbodiimide, N,N′-dicyclohexylcarbodiimide/N-hydroxysuccinimide, N,N′-carbonyldiimidazole, N,N′-thionyldiimidazole, triphenylphosphine/carbon tetrachloride, triphenylphosphine/diethyl azodicarboxylate, O-(benzotriazol-1-yl)-N,N′N′,N′-tetramethyluronium tetrafluoroborate or other amide coupling reagents, such as those described in Comprehensive Functional Group Transformations Vol. 5, pages 257 ff. (Pergamon, C. J. Moody) and in the references cited therein, optionally in the presence of a base such as potassium carbonate, N-ethyldiisopropylamine or N,N-dimethylaminopyridine or with a corresponding reactive derivative such as the anhydrides, esters or halides thereof, in a solvent such as methylene chloride, tetrahydrofuran, dioxane, dimethylsulfoxide, dimethylformamide or acetone, optionally in the presence of a reaction accelerator such as sodium or potassium iodide and preferably in the presence of a base such as sodium carbonate or potassium carbonate or in the presence of a tertiary organic base such as N-ethyldiisopropylamine or N-methylmorpholine, which may simultaneously also serve as solvent, conveniently at temperatures between 0° C. and 150° C., preferably at temperatures between 0° C. and 80° C.

b) In order to prepare a compound of general formula I wherein A denotes a straight-chain C₂₋₃-alkyl group optionally substituted by a C₁₋₃-alkyl group wherein the methylene group linked to the aromatic group or heteroaromatic group is replaced by an oxygen atom or —NH— group, while the —NH— group may additionally be substituted by a C₁₋₃-alkyl or carboxy-C₁₋₃-alkyl group:

reacting a compound of general formula

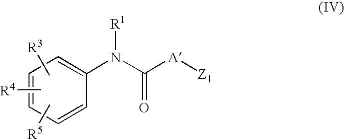

wherein:

R¹ and R³ to R⁵ are as hereinbefore defined,

A′ denotes a methylene or n-ethylene group optionally substituted by a C₁₋₃-alkyl group, and

Z₁ denotes a nucleofugic leaving group such as a halogen atom, e.g., a chlorine, bromine or iodine atom, or a p-nitrophenyloxy group,

with a compound of general formula

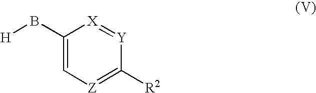

wherein X, Y, Z, and R² are as hereinbefore defined and B denotes an oxygen atom or an —NH— group optionally substituted by a C₁₋₃-alkyl or carboxy-C₁₋₃-alkyl group.

The reaction is preferably carried out in a solvent such as methylene chloride, acetonitrile, tetrahydrofuran, toluene, acetone/water, dimethylformamide or dimethylsulfoxide, optionally in the presence of a base such as sodium hydride, potassium carbonate, potassium tert-butoxide or N-ethyldiisopropylamine, at temperatures between 0° C. and 60° C.

c) In order to prepare a compound of general formula I wherein R² denotes an amidino group:

reacting a compound of general formula

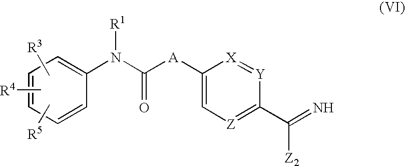

optionally formed in the reaction mixture, wherein R¹, R³ to R⁵, A, X, Y, and Z are as hereinbefore defined, and Z₂ denotes an alkoxy or aralkoxy group such as the methoxy, ethoxy, n-propoxy, isopropoxy or benzyloxy group or an alkylthio or aralkylthio group such as the methylthio, ethylthio, n-propylthio or benzylthio group,

with ammonia or hydroxylamine as well as with the salts thereof.

The reaction is expediently carried out in a solvent such as methanol, ethanol, n-propanol, tetrahydrofuran or dioxane at temperatures between 0° C. and 150° C., preferably at temperatures between 0° C. and 80° C., with ammonia or one of the salts thereof such as for example ammonium carbonate or ammonium acetate.

A compound of general formula VI is obtained for example by reacting a corresponding cyano compound with a corresponding alcohol such as methanol, ethanol, n-propanol, isopropanol or benzyl alcohol in the presence of an acid such as hydrochloric acid or by reacting a corresponding amide with a trialkyloxonium salt such as triethyloxonium tetrafluoroborate in a solvent such as methylene chloride, tetrahydrofuran or dioxane at temperatures between 0° C. and 50° C., but preferably at 20° C., or a corresponding nitrile with hydrogen sulfide, conveniently in a solvent such as pyridine or dimethylformamide and in the presence of a base such as triethylamine, and subsequent alkylation of the thioamide formed with a corresponding alkyl or aralkyl halide.

d) In order to prepare a compound of general formula I wherein R² denotes an amidino group substituted by a group which can be cleaved in vivo:

reacting a compound of general formula

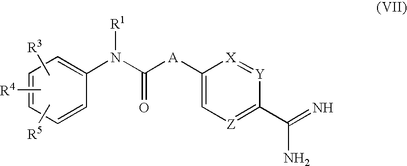

wherein R¹, R³ to R⁵, A, X, Y, and Z are as hereinbefore defined,

with a compound of general formula

Z₃—R⁶ (VIII)

wherein R⁶ denotes the acyl group of one of the groups which can be cleaved in vivo mentioned hereinbefore, and Z₃ denotes a nucleofugic leaving group such as a halogen atom, e.g., a chlorine, bromine or iodine atom, or a p-nitrophenyloxy group.

The reaction is preferably carried out in a solvent such as methanol, ethanol, methylene chloride, acetonitrile, tetrahydrofuran, toluene, acetone/water, dimethylformamide, or dimethylsulfoxide, optionally in the presence of an inorganic or a tertiary organic base such as sodium hydride, potassium carbonate, potassium tert-butoxide or N-ethyldiisopropylamine at temperatures between 0° C. and the boiling temperature of the solvent used, preferably at temperatures between 0° C. and 60° C.

e) In order to prepare a compound of general formula I wherein at least one of the groups A, R¹ or R³ contains a carboxy group:

converting a compound of general formula

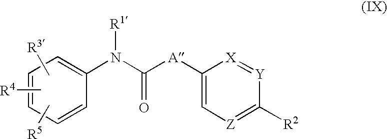

wherein R², R⁴, R⁵, X, Y, and Z are as hereinbefore defined and A″, R^{1′}, and R^{3′} have the meanings given hereinbefore for A, R¹, and R³, with the proviso that at least one of the groups A, R¹ or R³ contains a group which can be converted into a carboxy group by hydrolysis, treatment with an acid or base, thermolysis or hydrogenolysis,

into a compound of general formula I wherein at least one of the groups A, R¹ or R³ contains a carboxy group, by hydrolysis, treatment with an acid or base, thermolysis or hydrogenolysis.

By a group which may be converted into a carboxy group is meant for example a carboxyl group protected by a protecting group, such as the functional derivatives thereof, e.g., the unsubstituted or substituted amides, esters, thioesters, trimethylsilylesters, orthoesters or iminoesters thereof, which may conveniently be converted into a carboxyl group by hydrolysis,

the esters thereof with tertiary alcohols, e.g., the tert-butyl ester thereof, which may conveniently be converted into a carboxyl group by treatment with an acid or thermolysis, and

the esters thereof with aralkanols, e.g., the benzyl ester thereof, which may conveniently be converted into a carboxyl group by hydrogenolysis.

The hydrolysis is conveniently carried out either in the presence of an acid such as hydrochloric acid, sulfuric acid, phosphoric acid, acetic acid, trichloroacetic acid, trifluoroacetic acid or mixtures thereof or in the presence of a base such as lithium hydroxide, sodium hydroxide or potassium hydroxide in a suitable solvent such as water, water/methanol, water/ethanol, water/isopropanol, methanol, ethanol, water/tetrahydrofuran or water/dioxane at temperatures between −10° C. and 120° C., e.g., at temperatures between ambient temperature and the boiling temperature of the reaction mixture.

If a compound of general formula IX contains the tert-butyl or tert-butyloxycarbonyl group for example, these may also be cleaved by treatment with an acid such as trifluoroacetic acid, formic acid, p-toluenesulfonic acid, sulfuric acid, hydrochloric acid, phosphoric acid or polyphosphoric acid, optionally in an inert solvent such as methylene chloride, chloroform, benzene, toluene, diethyl ether, tetrahydrofuran or dioxane, preferably at temperatures between −10° C. and 120° C., e.g., at temperatures between 0° C. and 60° C., or also thermally, optionally in an inert solvent such as methylene chloride, chloroform, benzene, toluene, tetrahydrofuran, or dioxane, and preferably in the presence of a catalytic amount of an acid such as p-toluenesulfonic acid, sulfuric acid, phosphoric acid or polyphosphoric acid, preferably at the boiling temperature of the solvent used, e.g., at temperatures between 40° C. and 120° C.

If a compound of general formula IX contains the benzyloxy or benzyloxycarbonyl group, for example, these may also be cleaved by hydrogenolysis in the presence of a hydrogenation catalyst such as palladium/charcoal in a suitable solvent such as methanol, ethanol, ethanol/water, glacial acetic acid, ethyl acetate, dioxane or dimethylformamide, preferably at temperatures between 0° C. and 50° C., e.g., at ambient temperature, and a hydrogen pressure of 1 bar to 5 bar.

In the reactions described hereinbefore, any reactive groups present such as carboxy, amino or alkylamino groups may be protected during the reaction by conventional protecting groups which are cleaved again after the reaction.

For example, a protecting group for a carboxyl group may be a trimethylsilyl, methyl, ethyl, tert-butyl, benzyl or tetrahydropyranyl group, and

protecting groups for an amino or alkylamino group may be an acetyl, trifluoroacetyl, benzoyl, ethoxycarbonyl, tert-butoxycarbonyl, benzyloxycarbonyl, benzyl, methoxybenzyl or 2,4-dimethoxybenzyl group, and additionally, for the amino group, a phthalyl group.

Any protecting group used is optionally subsequently cleaved for example by hydrolysis in an aqueous solvent, e.g., in water, isopropanol/water, tetrahydrofuran/water or dioxane/water, in the presence of an acid such as trifluoroacetic acid, hydrochloric acid or sulfuric acid or in the presence of an alkali metal base such as lithium hydroxide, sodium hydroxide or potassium hydroxide, or by ether splitting, e.g., in the presence of iodotrimethylsilane, at temperatures between 0° C. and 100° C., preferably at temperatures between 10° C. and 50° C.

However, a benzyl, methoxybenzyl or benzyloxycarbonyl group is cleaved, for example, hydrogenolytically, e.g., with hydrogen in the presence of a catalyst such as palladium/charcoal in a solvent such as methanol, ethanol, ethyl acetate, dimethylformamide, dimethylformamide/acetone or glacial acetic acid, optionally with the addition of an acid such as hydrochloric acid at temperatures between 0° C. and 50° C., but preferably at ambient temperature, and at a hydrogen pressure of 1 bar to 7 bar, but preferably 3 bar to 5 bar.

A methoxybenzyl group may also be cleaved in the presence of an oxidizing agent such as cerium (IV) ammonium nitrate in a solvent such as methylene chloride, acetonitrile or acetonitrile/water at temperatures of between 0° C. and 50° C., but preferably at ambient temperature.

A 2,4-dimethoxybenzyl group, however, is preferably cleaved in trifluoroacetic acid in the presence of anisole.

A tert-butyl or tert-butyloxycarbonyl group is preferably cleaved by treating with an acid such as trifluoroacetic acid or hydrochloric acid, optionally using a solvent such as methylene chloride, dioxane or ether.

A phthalyl group is preferably cleaved in the presence of hydrazine or a primary amine such as methylamine, ethylamine or n-butylamine in a solvent such as methanol, ethanol, isopropanol, toluene/water or dioxane, at temperatures between 20° C. and 50° C.

An allyloxycarbonyl group is cleaved by treating with a catalytic amount of tetrakis-(triphenylphosphine)palladium(0), preferably in a solvent such as tetrahydrofuran and preferably in the presence of an excess of a base such as morpholine or 1,3-dimedone at temperatures between 0° C. and 100° C., preferably at ambient temperature and under an inert gas, or by treating with a catalytic amount of tris-(triphenylphosphine)rhodium(I) chloride in a solvent such as aqueous ethanol and optionally in the presence of a base such as 1,4-diazabicyclo[2.2.2]octane at temperatures between 20° C. and 70° C.

The compounds of general formulae II to IX used as starting materials, some of which are known from the literature, are obtained by methods known from the literature and the preparation thereof is also described in the Examples.

Thus, for example, a compound of general formula II is obtained by alkylation and/or acylation of a corresponding nitroaniline, and the nitroaniline thus obtained is then converted by reduction into a corresponding diamino compound and the resulting diamino compound is then converted if necessary by alkylation and/or acylation into the desired starting compound of general formula II. During these reactions any reactive group present such as an amino or imino group may optionally be protected by a conventional protecting group which is then cleaved again by conventional methods.

A starting compound of general formulae IV, VII, and IX is conveniently obtained analogously to method a) of the present invention.

Moreover, the compounds of general formula I obtained may be resolved into their enantiomers and/or diastereomers.

Thus, for example, the compounds of general formula I obtained which occur as racemates may be separated by methods known per se (cf. N. L. Allinger and E. L. Eliel in “Topics in Stereochemistry”, Vol. 6, Wiley Interscience, 1971) into their optical enantiomers and compounds of general formula I with at least 2 asymmetric carbon atoms may be resolved into their diastereomers on the basis of their physical-chemical differences using methods known per se, e.g., by chromatography and/or fractional crystallization, and, if these compounds are obtained in racemic form, they may subsequently be resolved into the enantiomers as mentioned above.

The enantiomers are preferably separated by column separation on chiral phases or by recrystallization from an optically active solvent or by reacting with an optically active substance which forms salts or derivatives such as, e.g., esters or amides with the racemic compound, particularly acids and the activated derivatives or alcohols thereof, and separating the diastereomeric mixture of salts or derivatives thus obtained, e.g., on the basis of their differences in solubility, whilst the free antipodes may be released from the pure diastereomeric salts or derivatives by the action of suitable agents. Optically active acids in common use are, e.g., the D- and L-forms of tartaric acid or dibenzoyltartaric acid, di-o-tolyltartaric acid, malic acid, mandelic acid, camphorsulfonic acid, glutamic acid, aspartic acid or quinic acid. An optically active alcohol may be, for example, (+) or (−)-menthol and an optically active acyl group in amides, for example, may be a (+)- or (−)-menthyloxycarbonyl.

Furthermore, the compounds of formula I may be converted into the salts thereof, particularly for pharmaceutical use into the physiologically acceptable salts with inorganic or organic acids. Acids which may be used for this purpose include for example hydrochloric acid, hydrobromic acid, sulfuric acid, methanesulfonic acid, phosphoric acid, fumaric acid, succinic acid, lactic acid, citric acid, tartaric acid or maleic acid.

Moreover, if the new compounds of formula I contain a carboxy group, they may subsequently, if desired, be converted into the salts thereof with inorganic or organic bases, particularly for pharmaceutical use into the physiologically acceptable salts thereof. Suitable bases for this purpose include for example sodium hydroxide, potassium hydroxide, cyclohexylamine, ethanolamine, diethanolamine, and triethanolamine.

As already mentioned, the new compounds of general formula I and the salts thereof have valuable properties. Thus, the compounds of general formula I wherein R² denotes a cyano group are valuable intermediate products for preparing the other compounds of general formula I, and the compounds of general formula I wherein R² denotes one of the amidino groups mentioned hereinbefore, as well as the tautomers, the stereoisomers, and the physiologically acceptable salts thereof have valuable pharmacological properties, particularly an antithrombotic activity, which is preferably based on a thrombin- or factor Xa-influencing activity, for example on a thrombin-inhibiting or factor Xa-inhibiting activity, on an aPTT-time-prolonging activity and on an inhibiting effect on related serine proteases such as, e.g., trypsin, urokinase factor VIIa, factor IX, factor XI, and factor XII.

For example, the compounds

A=4-{N-[2,5-dimethyl-4-(2-methylpyrrolidinocarbonyl)phenylaminocarbonylmethyl]-amino}benzamidine,

B=4-[N-(2,5-dimethyl-4-isopropylcarbonylphenylaminocarbonylmethyl)amino]benzamidine, and

C=4-{N-[2,5-dimethyl-4-(N′-isopropyl-N′-(2-ethoxycarbonyl-ethylcarbonyl)amino)phenylaminocarbonylmethyl]amino}benzamidine

were investigated for their effect on prolonging the aPTT time as follows:

Materials: plasma, from human citrated blood;

PTT reagent, Boehringer Mannheim (524298);

Calcium solution (0.025 mol/L), Behring Werke, Marburg (ORH 056/57),

Diethylbarbiturate acetate buffer, Behring Werke, Marburg (ORWH 60/61); and

Biomatic B10 coagulometer, Desaga, Wiesloch.

Method:

The aPTT time was determined using a Biomatic B10 coagulometer made by Messrs. Desaga.

The test substance was placed in the test tubes prescribed by the manufacturer with 0.1 mL of human citrated plasma and 0.1 mL of PTT reagent. The mixture was incubated for three minutes at 37° C. The clotting reaction was started by the addition of 0.1 mL of calcium solution. The time is measured using the apparatus from the addition of the calcium solution up to the clotting of the mixture. Mixtures to which 0.1 mL of DBA buffer had been added were used as the controls.

According to the definition, a dosage-activity curve was used to determine the effective concentration of the substance at which the aPTT time is double compared with the control.

The Table which follows contains the results found:

| | aPTT time |
|---|---|
| Substance | (ED₂₀₀ in μM) |
| A | 0.65 |
| B | 0.67 |
| C | 0.57 |

The compounds of general formula I prepared according to the invention wherein R² does not denote a cyano group are well tolerated as no toxic side effects could be observed in the pharmacological trials.

In view of their pharmacological properties the new compounds and the physiologically acceptable salts thereof are suitable for the prevention and treatment of venous and arterial thrombotic diseases, such as for example the treatment of deep leg vein thrombosis, for preventing reocclusions after bypass operations or angioplasty (PTCA), and occlusion in peripheral arterial diseases such as pulmonary embolism, disseminated intravascular coagulation, for preventing coronary thrombosis, stroke, and the occlusion of shunts. In addition, the compounds according to the invention are suitable for antithrombotic support in thrombolytic treatment, such as for example with alteplase, reteplase, tenecteplase, staphylokinase or streptokinase, for preventing long-term restenosis after PT(C)A, for the prevention and treatment of ischemic incidents in patients with unstable angina or non-transmural myocardial infarct, for preventing metastasization and the growth of coagulation-dependent tumors and fibrin-dependent inflammatory processes, e.g., in the treatment of pulmonary fibrosis, for preventing and treating rheumatoid arthritis, for preventing and treating fibrin-dependent tissue adhesions and/or the formation of scar tissue, and for promoting wound healing processes. The new compounds and the physiologically acceptable salts thereof may be used therapeutically in conjunction with inhibitors of platelet aggregation such as fibrinogen receptor antagonists (e.g., abciximab, eptifibatide, tirofiban), with inhibitors of ADP-induced aggregation (e.g., clopidogrel, ticlopidine), with P₂T receptor antagonists (e.g., cangrelor) or with combined thromboxane receptor antagonists/synthetase inhibitors (e.g., terbogrel).

The dosage required to achieve such an effect is appropriately 0.1 to 30 mg/kg, preferably 0.3 to 10 mg/kg by intravenous route, and 0.1 to 50 mg/kg, preferably 0.3 to 30 mg/kg by oral route, in each case administered 1 to 4 times a day. For this purpose, the compounds of formula I prepared according to the invention may be formulated, optionally together with other active substances, with one or more inert conventional carriers and/or diluents, e.g., with corn starch, lactose, glucose, microcrystalline cellulose, magnesium stearate, polyvinylpyrrolidone, citric acid, tartaric acid, water, water/ethanol, water/glycerol, water/sorbitol, water/polyethylene glycol, propylene glycol, cetylstearyl alcohol, carboxymethylcellulose or fatty substances such as hard fat or suitable mixtures thereof, to produce conventional galenic preparations such as plain or coated tablets, capsules, powders, suspensions or suppositories.

The Examples that follow are intended to illustrate the invention:

### EXAMPLE 1

### 4-[N-(5-phenylsulfonylamino-2-methylphenyl)aminocarbonylmethyl]benzamidine

### a. 5-phenylsulfonylamino-2-methylnitrobenzene

1.52 g (0.01 mol) 4-methyl-3-nitroaniline and 1.32 g (0.01 mol) benzenesulfonic acid chloride are heated in 30 mL of pyridine for 1 hour over a steam bath. The solvent is distilled off and the residue is purified by chromatography (silica gel; dichloromethane/ethanol 99:1). Yield: 2.9 g (100% of theory); R_{f} value: 0.29 (silica gel; dichloromethane/ethanol=50:1).

### b. 5-phenylsulfonylamino-2-methylaniline

2.9 g (0.01 mol) 5-phenylsulfonylamino-2-methylnitrobenzene is dissolved in 100 mL of methanol and after the addition of 0.5 g of 20% palladium on charcoal hydrogenated at ambient temperature with 5 bar hydrogen pressure. The catalyst is filtered off and the filtrate is concentrated by evaporation. Yield: 1.7 g (65% of theory); R_{f} value: 0.49 (silica gel; dichloromethane/ethanol=19:1); melting point: 142° C.-144° C.

### c. 4-[N-(5-phenylsulfonylamino-2-methylphenyl)aminocarbonylmethyl]benzonitrile

240 mg (1.5 mmol) of N,N′-carbonyldiimidazole is added to a solution of 242 mg (1.5 mmol) of 4-cyanophenylacetic acid in 30 mL of tetrahydrofuran and stirred for 15 minutes. Then 393 mg (1.5 mmol) of 5-phenylsulfonylamino-2-methylaniline is added and the mixture is stirred for 48 hours. The solvent is distilled off and the residue is purified by chromatography (silica gel; dichloromethane/ethanol=99:1). Yield: 0.53 g (87% of theory); R_{f} value: 0.25 (silica gel; dichloromethane/ethanol=50:1); melting point: 120° C.-122° C.

### d. 4-[N-(5-phenylsulfonylamino-2-methylphenylaminocarbonylmethyl]benzamidine

440 mg (1.09 mmol) of 4-[N-(5-phenylsulfonylamino-3-methylphenyl)aminocarbonyl-methyl]benzonitrile is stirred in 35 mL of ethanol saturated with hydrogen chloride gas at ambient temperature for 48 hours. The solvent is eliminated in vacuo at a maximum bath temperature of 30° C. and replaced with 35 mL of absolute ethanol. 1.05 g (11 mmol) of ammonium carbonate is added and the mixture is stirred for 48 hours. Then the solvent is distilled off and the residue is purified by chromatography (silica gel; dichloromethane/ethanol=9:1). Yield: 0.24 g (48% of theory); R_{f} value: 0.27 (silica gel; dichloromethane/ethanol=4:1); C₂₂H₂₂N₄O₃S×HCl (422.52/458.98); mass spectrum: (M+H)^{+—=423} and (2M+H)⁺=845.

### EXAMPLE 2

### 4-[N-(4-phenylsulfonylamino-2-methylphenyl)aminocarbonylmethyl]benzamidine

Prepared analogously to Example 1d from 4-[N-(4-phenylsulfonylamino-2-methylphenyl)aminocarbonylmethyl]benzonitrile, ethanol saturated with hydrogen chloride gas, and ammonium carbonate. Yield: 86% of theory; C₂₂H₂₂N₄O₃S×HCl (422.52/458.98); mass spectrum: (M+H)⁺=423.

### Example 3

### 4-[N-(2,3-dimethyl-5-phenylsulfonylaminophenyl)aminocarbonylmethyl]benzamidine

Prepared analogously to Example 1d from 4-[N-(2,3-dimethyl-5-phenylsulfonylaminophenyl)aminocarbonylmethyl]benzonitrile, ethanol saturated with hydrogen chloride gas, and ammonium carbonate. Yield: 82% of theory; R_{f} value: 0.24 (silica gel; dichloromethane/ethanol=4:1); C₂₃H₂₄N₄O₃S×HCl (436.54/473.01); mass spectrum: (M+H)⁺=437 and (2M+H)⁺=873.

### EXAMPLE 4

### 4-[N-(2,6-dichloro-4-phenylsulfonylaminophenyl)aminocarbonylmethyl]benzamidine

Prepared analogously to Example 1d from 4-[N-(2,6-dichloro-4-phenylsulfonylaminophenyl)aminocarbonylmethyl]benzonitrile, ethanol saturated with hydrogen chloride gas, and ammonium carbonate. Yield: 80% of theory; R_{f} value: 0.08 (silica gel; dichloromethane/ethanol=4:1); C₂₁H₁₈Cl₂N₄O₃S (477.39); mass spectrum: (M+H)⁺=477/79/81 (chlorine isotope) and (M+Na)⁺=499/01/03.

### EXAMPLE 5

### 4-[N-(2-ethyl-5-phenylsulfonylaminophenyl)aminocarbonylmethyl]benzamidine

Prepared analogously to Example 1d from 4-[N-(2-ethyl-5-phenylsulfonylaminophenyl)aminocarbonylmethyl]benzonitrile, ethanol saturated with hydrogen chloride gas, and ammonium carbonate. Yield: 52% of theory; R_{f} value: 0.32 (silica gel; dichloromethane/methanol=5:1); C₂₃H₂₄N₄O₃S×HCl (436.53/472.99); mass spectrum: (M+H)⁺=437 and (2M+H)⁺=873.

### EXAMPLE 6

### 4-[N-(6-phenylsulfonylaminoindan-4-yl)aminocarbonylmethyl]benzamidine

Prepared analogously to Example 1d from 4-[N-(6-phenylsulfonylaminoindan4-yl)amino-carbonylmethyl]benzonitrile, ethanol saturated with hydrogen chloride gas, and ammonium carbonate. Yield: 33% of theory; C₂₄H₂₄N₄O₃S×HCl (448.6/485.0); mass spectrum: (M+H)⁺=449.

### EXAMPLE 7

### 4-[N-(2,3-dimethyl-4-phenylsulfonylaminophenyl)aminocarbonylmethyl]benzamidine

Prepared analogously to Example 1d from 4-[N-(2,3-dimethyl-4-phenylsulfonylaminophenyl)aminocarbonylmethyl]benzonitrile, ethanol saturated with hydrogen chloride gas, and ammonium carbonate. Yield: 70% of theory; C₂₃H₂₄N₄O₃S×HCl (436.5/473.0); mass spectrum: (M+H)⁺=437.

### EXAMPLE 8

### 4-[N-(2,6-dimethyl-4-phenylsulfonylaminophenyl)aminocarbonylmethyl]benzamidine

Prepared analogously to Example 1d from 4-[N-(2,6-dimethyl-4-phenylsulfonylaminophenyl)aminocarbonylmethyl]benzonitrile, ethanol saturated with hydrogen chloride gas, and ammonium carbonate. Yield: 71% of theory; C₂₃H₂₄N₄O₃S×HCl (436.5/473.0); mass spectrum: (M+H)⁺=437 and (2M+H)⁺=873.

### EXAMPLE 9

### 4-[N-(2,6-dimethyl-3-phenylsulfonylaminophenyl)aminocarbonylmethyl]benzamidine

Prepared analogously to Example 1d from 4-[N-(2,6-dimethyl-3-phenylsulfonylaminophenyl)aminocarbonylmethyl]benzonitrile, ethanol saturated with hydrogen chloride gas, and ammonium carbonate. Yield: 67% of theory; C₂₃H₂₄N₄O₃S×HCl (436.5/473.0); mass spectrum: (M+H)⁺=437.

### EXAMPLE 10

### 4-{N-[2-methyl-5-(naphth-1-ylsulfonylamino)phenyl]aminocarbonylmethyl}benzamidine

Prepared analogously to Example 1d from 4-{N-[2-methyl-5-(naphth-1-ylsulfonylaminophenyl]aminocarbonylmethyl}benzonitrile, ethanol saturated with hydrogen chloride gas, and ammonium carbonate. Yield: 29% of theory; C₂₆H₂₄N₄O₃S×HCl (472.66/509.03); mass spectrum: (M+H)⁺=473.

### EXAMPLE 11

### 4-{N-[2-methyl-5-(3-methylphenylsulfonylamino)phenyl]aminocarbonylmethyl}benzamidine

Prepared analogously to Example 1d from 4-{N-[2-methyl-5-(3-methylphenylsulfonylamino)phenyl]aminocarbonylmethyl}benzonitrile, ethanol saturated with hydrogen chloride gas, and ammonium carbonate. Yield: 59% of theory; C₂₃H₂₄N₄O₃S×HCl (436.5/473.0); mass spectrum: (M+H)⁺=437.

### EXAMPLE 12

### 4-{N-[2-methyl-5-(2-trifluoromethylphenylsulfonylamino)phenyl]aminocarbonyl-methyl}benzamidine

Prepared analogously to Example 1d from 4-{N-[2-methyl-5-(2-fluoromethylphenylsulfonyl-amino)phenyl]aminocarbonylmethyl}benzonitrile, ethanol saturated with hydrogen chloride gas, and ammonium carbonate. Yield: 48% of theory; C₂₃H₂₁F₃N₄O₃S×HCl (490.5/526.97); mass spectrum: (M+H)⁺=491.

### EXAMPLE 13

### 4-{N-[2-methyl-5-(2-ethoxycarbonylphenylsulfonylamino)phenyl]aminocarbonyl-methyl}benzamidine

Prepared analogously to Example 1d from 4-{N-[2-methyl-5-(2-ethoxycarbonylphenylsulfonyl-amino)phenyl]aminocarbonylmethyl}benzonitrile, ethanol saturated with hydrogen chloride gas, and ammonium carbonate. Yield: 34% of theory; C₂₅H₂₆N₄O₅S×HCl (494.55/531.04); mass spectrum: (M+H)⁺=495 and (2M+H)⁺=989.

### EXAMPLE 14

### 4-{N-[5-(N′-benzyl-N′-butylaminocarbonyl)-2-methylphenyl]aminocarbonylmethyl}benzamidine

Prepared analogously to Example 1d from 4-{N-[5-(N′-benzyl-N′-butylaminocarbonyl)-2-methylphenyl]aminocarbonylmethyl}benzonitrile, ethanol saturated with hydrogen chloride gas, and ammonium carbonate. Yield: 39% of theory; C₂₈H₃₂N₄O₂×HCl (456.6/493.0); mass spectrum: (M+H)⁺=457.

### EXAMPLE 15

### 4-[N-(2,4-dimethyl-5-phenylsulfonylaminophenyl)aminocarbonylmethyl]benzamidine

Prepared analogously to Example 1d from 4-[N-(2,4-dimethyl-5-phenylsulfonylaminophenyl)aminocarbonylmethyl]benzonitrile, ethanol saturated with hydrogen chloride gas and ammonium carbonate. Yield: 85% of theory; C₂₃H₂₄N₄O₅S×HCl (436.54/473.01); mass spectrum: M⁺=436.

### EXAMPLE 16

### 4-{N-[2,4-dimethyl-5-(naphth-1-ylsulfonylamino)phenyl]aminocarbonylmethyl}benzamidine

Prepared analogously to Example 1d from 4-{N-[2,4-dimethyl-5-(naphth-1-ylsulfonylamino)phenyl]aminocarbonylmethyl}benzonitrile, ethanol saturated with hydrogen chloride gas and ammonium carbonate. Yield: 79% of theory; C₂₇H₂₆N₄O₅S×HCl (486.60/523.07); mass spectrum: (M+H)⁺=487.

### EXAMPLE 17

### 4-[N-(2,4-dimethyl-5-benzylsulfonylaminophenyl)aminocarbonylmethyl]benzamidine

Prepared analogously to Example 1d from 4-[N-(2,4-dimethyl-5-benzylsulfonylaminophenyl)aminocarbonylmethyl]benzonitrile, ethanol saturated with hydrogen chloride gas and ammonium carbonate. Yield: 78% of theory; C₂₄H₂₆N₄O₅S×HCl (450.57/487.04); mass spectrum: M⁺=450.

### EXAMPLE 18

### 4-[N-(2-ethoxycarbonylmethyl-5-phenylsulfonylaminophenyl)aminocarbonylmethyl]benzamidine

### a. tert-butyl 2,4-diaminophenylacetate

Prepared analogously to Example 1b from tert-butyl 2,4-dinitrophenylacetate, 10% palladium on charcoal and hydrogen in methanol. Yield: 94% of theory; R_{f} value: 0.36 (silica gel; dichloromethane/methanol=19:1).

### b. tert-butyl 2-amino-4-phenylsulfonylaminophenylacetate

Prepared analogously to Example 1a from tert-butyl 2,4-diaminophenylacetate and benzenesulfonic acid chloride in pyridine. Yield: 30% of theory; R_{f} value: 0.34 (silica gel; dichloromethane/methanol=19:1).

### c. 4-[N-(2-ethoxycarbonylmethyl-5-phenylsulfonylaminophenyl)aminocarbonyl-methyl]benzamidine

Prepared analogously to Example 1c from tert-butyl 2-amino-4-phenylsulfonylaminophenylacetate, 4-cyanophenylacetic acid, and N,N′-carbonyldiimidazole in tetrahydrofuran at 50° C., subsequently reacting analogously to Example 1d with ethanol saturated with hydrogen chloride gas and ammonium carbonate. Yield: 56% of theory; C₂₅H₂₆N₄O₅S×HCl (494.56/531.03); mass spectrum: (M+H)⁺=495.

### EXAMPLE 19

### 4-[N-(2-trifluoromethyl-4-phenylsulfonylaminophenyl)aminocarbonylmethyl]benzamidine

Prepared analogously to Example 1d from 4-[N-(2-trifluoromethyl-4-phenylsulfonylaminophenyl)aminocarbonylmethyl]benzonitrile, ethanol saturated with hydrogen chloride gas, and ammonium carbonate. Yield: 46% of theory; C₂₂H₁₉F₃N₄O₃S×HCl (476.48/512.94); mass spectrum: (M+H)⁺=477.

### EXAMPLE 20

### 4-{N-[2-methyl-5-(2,3,5,6-tetramethylphenylsulfonylamino)phenyl]aminocarbonyl-methyl}benzamidine

Prepared analogously to Example 1d from 4-{N-[2-methyl-5-(2,3,5,6-tetramethylphenyl-sulfonylamino)phenyl]aminocarbonylmethyl}benzonitrile, ethanol saturated with hydrogen chloride gas, and ammonium carbonate. Yield: 79% of theory; C₂₆H₃₀N₄O₃S×HCl (478.61/515.07); R_{f} value: 0.18 (silica gel; dichloromethane/ethanol=4:1); mass spectrum: (M+H)⁺=479.

### EXAMPLE 21

### 4-[N-(2-hydroxycarbonylmethyl-5-phenylsulfonylaminophenylaminocarbonyl-methyl]benzamidine

A mixture of 0.53 g (0.001 mol) 4-[N-(2-ethoxycarbonylmethyl-5-phenyl-sulfonylaminophenyl)aminocarbonylmethyl]benzamidine, 0.2 g (0.005 mol) sodium hydroxide, 15 mL of water, and 3 mL of ethanol is stirred for 2 hours at ambient temperature. Then it is diluted with 10 mL of water and neutralized with 2 molar hydrochloric acid. The crystalline product is suction filtered, dissolved in 10 mL of tetrahydrofuran and 3 mL of water, adjusted to pH 4 with 2 molar hydrochloric acid, and concentrated by evaporation. Yield: 0.31 g (62% of theory); C₂₃H₂₂N₄O₅S×HCl (466.51/502.97); mass spectrum: (M+H)⁺=467 and (M+Na)⁺=489.

### EXAMPLE 22

### 4-{N-[5-(N′-(ethoxycarbonylmethyl)-N′-(naphth-1-ylsulfonyl)amino)-2-methylphenyl]aminocarbonylmethyl}benzamidine

### a. 5-[N-ethoxycarbonylmethyl-N-(naphth-1-ylsulfonyl)amino]-2-methylnitrobenzene

First 2 g (0.018 mol) of potassium tert-butoxide and, after 30 minutes, 2.5 mL (0.023 mol) of ethyl bromoacetate is added to a solution of 6 g (0.017 mol) 2-methyl-5-(naphthyl-1-ylsulfonyl)aminonitrobenzene in 120 mL of dimethylsulfoxide and the mixture is stirred for 12 hours. Then the reaction mixture is diluted with ether, washed with 14% sodium chloride solution, and dried. The solvent is distilled off and the residue is purified by chromatography (silica gel; dichloromethane/ethanol=99:1). Yield: 6.7 g (92% of theory); R_{f} value: 0.64 (silica gel; dichloromethane/ethanol=50:1).

### b. 5-[N-ethoxycarbonylmethyl-N-(naphth-1-ylylsulfonyl)amino]-2-methylaniline

Prepared analogously to Example 1b from 5-[N-ethoxycarbonylmethyl-N-(naphth-1-ylsulfonyl)amino]-2-methylnitrobenzene, 10% palladium on charcoal and hydrogen in methanol. Yield: 99% of theory; R_{f} value: 0.46 (silica gel; dichloromethane/ethanol=19:1).

### c. 4-{N-[5-(N′-(ethoxycarbonylmethyl)-N′-(naphth-1-ylsulfonyl)amino)-2-methylphenyl]aminocarbonylmethyl}benzamidine

Prepared analogously to Example 1c from 5-[N-ethoxycarbonylmethyl-N-(naphth-1-ylsulfonyl)amino]-2-methylaniline, 4-cyanophenylacetic acid, and N,N′-carbonyldiimidazole in tetrahydrofuran, subsequently reacted analogously to Example 1d with ethanol saturated with hydrogen chloride gas and ammonium carbonate. Yield: 39% of theory, C₃₀H₃₀N₄O₅S×HCl (558.7/595.1); mass spectrum: (M+H)⁺=559.

### EXAMPLE 23

### 4-{N-[5-(N′-(morpholinocarbonylmethyl)-N′-(naphth-1-ylsulfonyl)amino)-2-methylphenyl]aminocarbonylmethyl}benzamidine

Prepared analogously to Example 1d from 4-{N-[5-(N′-(morpholinocarbonylmethyl)-N′-(naphth-1-ylsulfonyl)amino)-2-methylphenyl]aminocarbonylmethyl}benzonitrile, ethanol saturated with hydrogen chloride gas, and ammonium carbonate. Yield: 13% of theory, C₃₂H₃₃N₅O₅S×HCl (599.7/636.18); mass spectrum: (M+H)⁺=600.

### EXAMPLE 24

### 4-{N-[5-(N′-methyl-N′-(naphth-1-ylsulfonyl)amino)-2-methylphenyl]aminocarbonyl-methyl}benzamidine

Prepared analogously to Example 1d from 4-{N-[5-(N′-methyl-N′-(naphth-1-ylsulfonyl)amino)-2-methylphenyl]aminocarbonylmethyl}benzonitrile, ethanol saturated with hydrogen chloride gas, and ammonium carbonate. Yield: 51% of theory; C₂₇H₂₆N₄O₃S×CH₃COOH (486.6/546.6); mass spectrum: (M+H)⁺=487.

### EXAMPLE 25

### 4-{N-[5-(N′-hydroxycarbonylmethyl-N′-(naphth-1-ylsulfonyl)amino)-2-methylphenyl]aminocarbonylmethyl}benzamidine

Prepared analogously to Example 21 from 4-{N-[5-(N′-ethoxycarbonylmethyl-N′-(naphth-1-ylsulfonyl)amino)-2-methylphenyl]aminocarbonylmethyl}benzamidine, sodium hydroxide solution in ethanol/water, and subsequent treatment with hydrochloric acid. Yield: 87% of theory; C₂₈H₂₆N₄O₅S×HCl (530.6/567.1); mass spectrum: (M+H)⁺=531.

### EXAMPLE 26

### 4-[N-(2-methyl-5-phenylaminosulfonylphenyl)aminocarbonylmethyl]benzamidine

Prepared analogously to Example 1d from 4-[N-(2-methyl-5-phenylaminosulfonylphenyl)aminocarbonylmethyl]benzonitrile, ethanol saturated with hydrogen chloride gas, and ammonium carbonate. Yield: 17% of theory; C₂₂H₂₂N₄O₃S×HCl (422.5/458.97); mass spectrum: (M+H)⁺=423.

### EXAMPLE 27

### 4-{N-[2-methyl-4-(quinolin-8-ylsulfonylamino)phenyl]aminocarbonylmethyl}benzamidine

Prepared analogously to Example 1d from 4-{N-[2-methyl-4-(quinolin-8-ylsulfonylamino)phenyl]aminocarbonylmethyl}benzonitrile, ethanol saturated with hydrogen chloride gas, and ammonium carbonate. Yield: 26% of theory; C₂₅H₂₃N₅O₃S×HCl (473.6/510.1); mass spectrum: (M+H)⁺=474 and (2M+H)⁺=947.

### EXAMPLE 28

### 4-{N-[2-methyl-5-(1,2,3,4-tetrahydroquinolin-8-ylsulfonylamino)phenyl]amino-carbonylmethyl}benzamidine

Prepared analogously to Example 1d from 4-{N-[2-methyl-5-(1,2,3,4-tetrahydroquinolin-8-ylsulfonylamino)phenyl]aminocarbonylmethyl}benzonitrile, ethanol saturated with hydrogen chloride gas, and ammonium carbonate. Yield: 43% of theory; C₂₅H₂₇N₅O₃S×2 HCl (477.6/550.52); mass spectrum: (M+H)⁺=478.

### EXAMPLE 29

### 4-{N-(4-benzylphenyl)-N-ethylaminocarbonylmethyloxy}benzamidine

Prepared analogously to Example 1d from 4-{N-(4-benzylphenyl)-N-ethylaminocarbonyl-methyloxy}benzonitrile, ethanol saturated with hydrogen chloride gas, and ammonium acetate.

Yield: 33% of theory; C₂₄H₂₅N₃O₂×HI (387.48/515.40); mass spectrum: (M+H)⁺=388.

### EXAMPLE 30

### 4-{2-{N-[4-(N′-ethoxycarbonylmethyl-N′-(naphth-1-ylsulfonyl)amino)phenyl]-N-ethylaminocarbonyl}ethyl}benzamidine

### a. 4-(naphth-1-ylsulfonylamino)nitrobenzene

10 g (0.042 mol) naphthalene-4-sulfonic acid chloride is added to a solution of 5.8 g (0.042 mol) of 4-nitrobenzene in 20 mL of pyridine while cooling with an ice bath and warmed to a bath temperature of 100° C. within 20 minutes. After another 20 minutes, the mixture is cooled to 50° C., 8 mL of 6 molar sodium hydroxide solution are added and then the mixture is stirred for 20 minutes at 60° C. The solvent is distilled off, the residue is stirred into 150 mL of water, and the product is suction filtered. Yield: 11.2 g (81% of theory); melting point: 210° C.-215° C.

### b. 4-[N-ethoxycarbonylmethyl-N-(naphth-1-ylsulfonyl)amino]nitrobenzene

3.9 g (39 mmol) of potassium tert-butoxide is added to a solution of 11.1 g (33.8 mmol) of 4-(naphthalene-1-sulfonylamino)nitrobenzene in 100 mL of dimethylformamide at 0° C. and, after 1 hour, 7 g (42 mmol) of ethyl bromoacetate are added. Then the mixture is stirred for 1.5 hours at 0° C. and for 12 hours at ambient temperature. It is then diluted with ethyl acetate and washed with water. The organic phase is dried and concentrated by evaporation. The residue is purified by chromatography (silica gel; methylene chloride).Yield: 14 g (100% of theory); R_{f} value: 0.43 (silica gel; dichloromethane).

### c. 4-[N-ethoxycarbonylmethyl-N-(naphth-1-ylsulfonyl)amino]aniline

Prepared analogously to Example 1b from 4-[N-ethoxycarbonylmethyl-N-(naphth-1-ylsulfonyl)amino]nitrobenzene, 10% palladium on charcoal, and hydrogen in ethanol. Yield: 82% of theory; melting point: 98° C.-103° C.; C₂₀H₂₀N₂O₄S (384.45); mass spectrum: M⁺=384.

### d. N-[4-(N′-ethoxycarbonylmethyl-N′-(naphth-1-ylsulfonyl)amino)phenyl]ethylamine

0.48 mL (84 mmol) of acetaldehyde and 0.48 mL glacial acetic acid are added to a solution of 3.2 g (84 mmol) of 4-[N-ethoxycarbonylmethyl-N-(naphth-1-ylsulfonyl)amino]aniline in 100 mL of methanol at 0° C. and then 0.53 g (84 mmol) of sodium cyanoborohydride are added batchwise. The reaction is then allowed to come up to ambient temperature and stirred for a further 5 hours. The solvent is distilled off, the residue is taken up in ethyl acetate and washed with water. The combined organic extracts are dried and concentrated by evaporation. Yield: 3.3 g (95% of theory); R_{f} value: 0.36 (silica gel; dichloromethane/ethyl acetate=19:1).

### e. 4-{2-{N-[4-(N′-ethoxycarbonylmethyl-N′-(naphth-1-ylsulfonyl)amino)phenyl]-N-ethylaminocarbonyl}ethyl}benzonitrile

0.96 mL (86 mmol) of N-methylmorpholine and 1 mL (79 mmol) of isobutyl chloroformate are added to a solution of 1.3 g (72 mmol) of 4-cyanophenylpropionic acid in 70 mL of tetrahydrofuran at −40° C. and stirred for 1 hour. Then at 0° C. 3.4 g (82 mmol) of N-[4-(N′-ethoxycarbonylmethyl-N′-(naphth-1-ylsulfonyl)amino)phenyl]ethylamine is added and the reaction is left overnight to come up to ambient temperature. It is then diluted with ethyl acetate and washed with water. The combined organic extracts are dried and concentrated by evaporation. The residue is purified by chromatography (silica gel; dichloromethane/ethyl acetate=19:1). Yield: 1.3 g (32% of theory).

### f. 4-{2-{N-[4-(N′-ethoxycarbonylmethyl-N′-(naphth-1-ylsulfonyl)amino)phenyl]-N-ethylaminocarbonyl}ethyl}benzamidine

Hydrogen sulfide is piped into a solution of 1.25 g (2.2 mmol) of 4-{2-{N-[4-(N′-ethoxycarbonylmethyl-N′-(naphth-1-ylsulfonyl)amino)phenyl]-N-ethylaminocarbonyl}ethyl}benzonitrile and 0.66 g (7 mmol) of triethylamine in 30 mL of pyridine for 20 minutes at 0° C. and stirred for 20 minutes. Then the solvent is distilled off, the residue is taken up in dichloromethane and washed with water. The organic phase is dried and concentrated by evaporation. The residue is taken up in 40 mL of acetone and combined with 3.1 g (0.022 mol) of methyl iodide. After 48 hours, it is concentrated by evaporation, the crude product is taken up in 50 mL of ethanol, combined with 0.9 g (0.012 mol) ammonium acetate, and stirred for 20 hours at ambient temperature. After the solvent has evaporated off, the residue is purified by chromatography (silica gel; dichloromethane/ethanol=9:1). Yield: 0.5 g (31% of theory); melting point: 75° C.-79° C.; C₃₂H₃₄N₄O₅S×HI (586.71/714.62); mass spectrum: (M+H)⁺=587.

### EXAMPLE 31

### 4-{2-{N-[4-(N′-hydroxycarbonylmethyl-N′-(naphth-1-ylsulfonyl)amino)phenyl]-N-ethylaminocarbonyl}ethyl}benzamidine

Prepared analogously to Example 21 from 4-{2-{N-[4-(N′-ethoxycarbonylmethyl-N′-(naphth-1-ylsulfonyl)amino)phenyl]-N-ethylaminocarbonyl}ethyl}benzamidine, sodium hydroxide solution in ethanol, and subsequent treatment with hydrochloric acid. Yield: 50% of theory; melting point: 191° C.-195° C.; C₃₀H₃₀N₄O₅S×HCl (558.66/595.12); mass spectrum: (M+H)⁺=559 and (M+Na)⁺=581.

### EXAMPLE 32

### 4-{N-[2,5-dimethyl-4-(N′-ethoxycarbonylmethylaminocarbonyl-N′-isopropylamino)phenyl]aminocarbonylmethyl}benzamidine

Prepared analogously to Example 1d from 4-{N-[2,5-dimethyl-4-(N′-ethoxycarbonylmethyl-aminocarbonyl-N′-isopropylamino)phenyl]aminocarbonylmethyl}benzonitrile, ethanol saturated with hydrogen chloride gas, and ammonium acetate. Yield: 64% of theory; C₂₅H₃₃N₅O₄×HCl (467.57/504.03); mass spectrum: (M+H)⁺=468 and (M+Cl)⁻=502/04 (chlorine isotope).

### EXAMPLE 33

### 4-{2-{N-[2,5-dimethyl-4-(N′-ethoxycarbonylmethylaminocarbonyl-N′-isopropylamino)phenyl]aminocarbonyl}ethyl}benzamidine

Prepared analogously to Example 1d from 4-{2-{N-[2,5-dimethyl-4-(N′-ethoxycarbonylmethylaminocarbonyl-N′-isopropylamino)phenyl]aminocarbonyl}ethyl}benzonitrile, ethanol saturated with hydrogen chloride gas, and ammonium acetate. Yield: 73% of theory; C₂₆H₃₅N₅O₄×HCl (481.59/518.06); mass spectrum: (M+H)⁺=482 and (M−H)⁻=480.

### EXAMPLE 34

### 4-{N-[4-phenylsulfonylaminophenyl]-N-ethylaminocarbonylmethylamino}benzamidine

Prepared analogously to Example 1d from 4-{N-[4-phenylsulfonylaminophenyl]-N-ethylaminocarbonylmethylamino}benzonitrile, ethanol saturated with hydrogen chloride gas, and ammonium carbonate. Yield: 54% of theory; C₂₃H₂₅N₅O₃S×HCl (451.55/488.01); mass spectrum: (M+H)⁺=452.

### EXAMPLE 35

### 4-{N-[N′-ethyl-N′-(4-piperidinomethylphenyl)aminocarbonylmethyl]-N-ethoxycarbonylmethylamino}benzamidine

### a. N-ethyl-4-piperidinomethylaniline

10 mL formaldehyde (37% in water) is added dropwise to 8.5 g (0.1 mol) of piperidine while cooling with ice and then first 12.1 g (0.1 mol) of N-ethylaniline and then 3 g (0.05 mol) of glacial acetic acid are added dropwise at 15° C. Then 40 mL of ethanol are added and the mixture is refluxed for 18 hours. The ethanol is then distilled off, the residue is taken up in dichloromethane and washed with dilute sodium hydroxide solution. The organic phase is dried and purified by chromatography (silica gel; ethyl acetate). Yield: 6.4 g (30% of theory); R_{f} value: 0.35 (silica gel; ethyl acetate/ethanol/ammonia=9:1:0.1).

### b. 4-{N-[N′-ethyl-N′-(4-piperidinomethylphenyl)aminocarbonylmethyl]-N-ethoxy-carbonylmethylamino}benzamidine

Prepared analogously to Example 30e from N-ethyl-4-piperidinomethylaniline, 4-(N-hydroxycarbonylmethyl-N-methoxycarbonyl)aminobenzonitrile, N-methylmorpholine, and isobutyl chloroformate in tetrahydrofuran. Yield: 58% of theory; R_{f} value: 0.74 (silica gel; dichloromethane/ethyl acetate=9:1).

### c. 4-{N-[N′-ethyl-N′-(4-piperidinomethylphenyl)aminocarbonylmethyl]-N-ethoxy-carbonylmethylamino}benzamidine

Prepared analogously to Example 1d from 4-{N-[ethyl-(4-piperidinomethylphenyl)amino-carbonylmethyl]-methoxycarbonylmethyl}aminobenzonitrile, ethanol saturated with hydrogen chloride gas, and ammonium acetate. Yield: 52% of theory; C₂₇H₃₇N₅O₃×2 HCl (479.63/552.55); mass spectrum: (M+H)⁺=480.

### EXAMPLE 36

### 4-{N-[N′-ethyl-N′-(3-benzylphenyl)aminocarbonylmethyl]-N-ethoxycarbonylmethylamino}benzamidine

prepared analogously to Example 1d from 4-{N-[′-ethyl-N′-(3-benzylphenyl)amino-carbonylmethyl]-N-ethoxycarbonylmethylamino}benzonitrile, ethanol saturated with hydrogen chloride gas, and ammonium acetate. Yield: 47% of theory; C₂₈H₃₂N₄O₃×HCl (472.59/509.05); Mass spectrum: (M+H)⁺=473.

### EXAMPLE 37

### 4-{N-[N′-ethyl-N′-(4-(pyridin-3-ylmethyl)phenyl)aminocarbonylmethyl]-N-ethoxy-carbonylmethylamino}benzamidine

Prepared analogously to Example 1d from 4-{N-[N′-ethyl-N′-(4-(pyridin-3-ylmethyl)phenyl)aminocarbonylmethyl]-N-ethoxycarbonylmethylamino}benzonitrile, ethanol saturated with hydrogen chloride gas, and ammonium acetate. Yield: 51% of theory; C₂₇H₃₁N₅O₃×HCl (473.54/510.04); mass spectrum: (M+H)⁺=474.

### EXAMPLE 38

### 4-{N-[4-(3,5-diethylpyrazol-1-yl)-3-methylphenylaminocarbonylmethyl]amino}benzamidine

### a. 4-(3,5-diethylpyrazol-1-yl)-3-methyliodobenzene

A mixture of 0.7 g (5.07 mmol) of heptane-3,5-dione, 1.4 g (5.06 mmol) of 4-hydrazino-3-methyliodobenzene, and 0.7 mL (5.1 mmol) of triethylamine are stirred in 40 mL of methanol for 3 hours at ambient temperature. The solvent is distilled off, the residue is taken up in 100 mL ether, washed with 50 mL 1N hydrochloric acid, dried, and concentrated by evaporation. The crude product is purified by chromatography (silica gel; dichloromethane/ethanol=99:1 to 97:3).Yield: 1.1 g (64% of theory); C₁₄H₁₇IN₂ (340.21); mass spectrum: (M+H)⁺=341 and (2M+H)⁺=681.

### b. 4-(3,5-diethylpyrazol-1-yl)-3-methyl-N-benzylaniline

A mixture of 1.1 g (3.2 mmol) of 4-(3,5-diethylpyrazol-1-yl)-3-methyliodobenzene, 0.5 mL (4.57 mmol) of benzylamine, 44 mg (0.19 mmol) of palladium (II) acetate, 120 mg (0.19 mmol) of 2,2′-bis-(diphenylphosphino)-1,1′-binaphthyl, and 0.6 g (6.4 mmol) of sodium tert-butoxide are stirred in 50 mL dioxane for 2 hours at 100° C. Then the solvent is distilled off and the residue is purified by chromatography (silica gel; dichloromethane/ethanol=49:1). Yield: 0.7 g (68% of theory); C₂₁H₂₅N₃ (319.45); mass spectrum: (M+H)⁺=320, (M−H)⁻=318, and (2M+Na)⁺=661.

### c. 4-(3,5-diethylpyrazol-1-yl)-3-methylaniline

0.7 g (2.2 mmol) of 4-(3,5-diethylpyrazol-1-yl)-3-methyl-N-benzylaniline is dissolved in 40 mL of methanol and after the addition of 0.5 g of palladium hydroxide on charcoal hydrogenated for 4 hours at ambient temperature with 3 bar hydrogen pressure. The catalyst is filtered off and the filtrate is concentrated by evaporation. Yield: 0.2 g (48% of theory); C₁₄H₁₉N₃ (229.33); mass spectrum: (M+H)⁺=230 and (M+Na)⁺=252.

### d. N-[4-(3,5-diethylpyrazol-1-yl)-3-methylphenyl]aminocarbonylmethyl bromide

0.2 g (1 mmol) of 4-(3,5-diethylpyrazol-1-yl)-3-methylaniline is added dropwise at 0° C. to a solution of 0.1 mL (1 mmol) of bromoacetyl chloride and 0.2 g (1.5 mmol) of potassium carbonate in 15 mL dioxane and 15 mL of water. After 10 minutes, the solvent is distilled off, the residue is taken up in 200 mL of ethyl acetate and 50 mL of water, the organic phase is separated off, dried, and concentrated by evaporation. Yield: 0.4 g (100% of theory).

### e. 4-{N-[4-(3,5-diethylpyrazol-1-yl)-3-methylphenylaminocarbonylmethyl]amino}benzonitrile

A mixture of 0.3 g (0.86 mmol) of N-[4-(3,5-diethylpyrazol-1-yl)-3-methylphenyl]-aminocarbonylmethyl bromide and 0.2 g (1.28 mmol) of 4-cyanoaniline is stirred for 3 hours in 15 mL of N-ethyldiisopropylamine at 100° C. Then the reaction mixture is concentrated and purified by chromatography. Yield: 40 mg (12% of theory); C₂₃H₂₃N₅O (387.49); mass spectrum: (M−H)⁻=386.

### f. 4-{N-[4-(3,5-diethylpyrazol-1-yl)-3-methylphenylaminocarbonylmethyl]amino}benzamidine

Prepared analogously to Example 1d from 4-{N-[4-(3,5-diethylpyrazol-1-yl)-3-methylphenyl-aminocarbonylmethyl]amino}benzonitrile, ethanol saturated with hydrogen chloride gas, and ammonium acetate. Yield: 79% of theory; C₂₃H₂₈N₆O×HCl (404.43/440.98); mass spectrum: (M+H)⁺=405.

### EXAMPLE 39

### 4-{N-[3-methyl-4-(pyrrolidinocarbonyl)phenylaminocarbonylmethyl]amino}benzamidine

Prepared analogously to Example 1d from 4-{N-[3-methyl-4-(pyrrolidinocarbonyl)phenyl-aminocarbonylmethyl]amino}benzonitrile, ethanol saturated with hydrogen chloride gas, and ammonium acetate. Yield: 59% of theory; C₂₁H₂₅N₅O₂×HCl (379.47/415.93); mass spectrum: (M+H)⁺=380.

### EXAMPLE 40

### 4-{N-[N′-methyl-3-methyl-4-(pyrrolidinocarbonyl)phenylaminocarbonylmethyl]-amino}benzamidine

Prepared analogously to Example 1d from 4-{N-[N′-methyl-3-methyl-4-(pyrrolidinocarbonyl)phenylaminocarbonylmethyl]amino}benzonitrile, ethanol saturated with hydrogen chloride gas, and ammonium acetate. Yield: 59% of theory; C₂₂H₂₇N₅O₂×HCl (393.49/429.95); mass spectrum: (M+H)⁺=394 and (M−H+HCl)⁻=428/30 (chlorine isotope).

### EXAMPLE 41

### 4-{N-methyl-N-[4-(N′-methyl-N′-(pyridin-2-yl)aminocarbonyl)-3-methylphenylamino-carbonylmethyl]amino}benzamidine

Prepared analogously to Example 1d from 4-{N-methyl-N-[4-(N′-methyl-N′-(pyridin-2-yl)aminocarbonyl)-3-methylphenylaminocarbonylmethyl]amino}benzonitrile, ethanol saturated with hydrogen chloride gas, and ammonium acetate. Yield: 21% of theory; C₂₄H₂₆N₆O₂×HCl (430.51/466.98); mass spectrum: (M+H)⁺=431.

### EXAMPLE 42

### 4-{N-[2,5-dimethyl-4-(2-methylpyrrolidinocarbonyl)phenylaminocarbonylmethyl]-amino}benzamidine

Prepared analogously to Example 1d from 4-{N-[2,5-dimethyl-4-(2-methylpyrrolidino-carbonyl)phenylaminocarbonylmethyl]amino}benzonitrile, ethanol saturated with hydrogen chloride gas, and ammonium acetate. Yield: 100% of theory; C₂₃H₂₉N₅O₂×HCl (407.52/443.98); mass spectrum: (M−H+HCl)⁻=442/44 (chlorine isotope).

### EXAMPLE 43

### 4-{N-[2,5-dimethyl-4-(N′-methyl-N′-phenylaminocarbonyl)phenylaminocarbonyl-methyl]amino}benzamidine

Prepared analogously to Example 1d from 4-{N-[2,5-dimethyl-4-(N′-methyl-N′-phenyl-aminocarbonyl)phenylaminocarbonylmethyl]amino}benzonitrile, ethanol saturated with hydrogen chloride gas, and ammonium acetate. Yield: 36% of theory; C₂₅H₂₇N₅O₂×HCl (429.53/465.99); mass spectrum: (M+H)⁺=430.

### EXAMPLE 44

### 4-[N-(2,5-dimethyl-4-isopropylcarbonylphenylaminocarbonylmethyl)amino]benzamidine

Prepared analogously to Example 1d from 4-[N-(2,5-dimethyl-4-isopropylcarbonylphenylaminocarbonylmethyl)amino]benzonitrile, ethanol saturated with hydrogen chloride gas, and ammonium acetate. Yield: 30% of theory; C₂₁H₂₆N₄O₂×HCl (366.47/402.93); mass spectrum: (M+H)⁺=367.

### EXAMPLE 45

### 4-{N-[2,5-dimethyl-4-isobutylphenylaminocarbonylmethyl]amino}benzamidine

2,5-dimethyl-4-isobutylaniline is obtained as a non-separable by-product when 2,5-dimethyl-4-isopropylcarbonyl-N-benzylaniline is reacted to form 2,5-dimethyl-4-isopropylcarbonylaniline analogously to Example 38c. The mixture was converted into the corresponding amide analogously to Example 1c with 4-cyanophenylaminoacetic acid and N,N′-carbonyldiimidazole in tetrahydrofuran and then reacted analogously to Example 1d to obtain a mixture of 4-{N-[2,5-dimethyl-4-isopropylcarbonylphenylaminocarbonylmethyl]amino}benzamidine and 4-{N-[2,5-dimethyl-4-isobutylphenyl]aminocarbonylmethylamino}benzamidine, which is purified by HPLC (Inertsil ODS2, 250 mm×10 mm, 5 μm, 0.1% KH₂PO₄/methanol, retention time of the title compound: 21.25 minutes). C₂₁H₂₈N₄O×HCl (352.48/388.49); mass spectrum: (M+H)⁺=353.

### EXAMPLE 46

### 4-{N-[2,5-dimethyl-4-(N′-isopropyl-N′-(2-ethoxycarbonylethyl-carbonyl)amino)phenylaminocarbonylmethyl]amino}benzamidine of formula

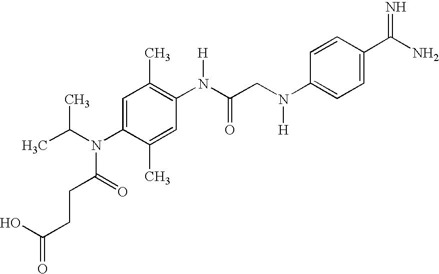

### a. 2,5-dimethyl-4-isopropylaminobenzyloxycarbonylaniline

0.7 mL (11.6 mmol) of glacial acetic acid and 0.1 g (1 mmol) of p-toluenesulfonic acid are added to a solution of 2.1 g (7.7 mmol) of 4-amino-2,5-dimethylbenzyloxycarbonylaniline and 0.6 mL (8.5 mmol) of acetone in 30 mL of tetrahydrofuran and stirred for 30 minutes. Then 2.3 g (10.1 mmol) of sodium triacetoxyborohydride is added and the mixture is stirred for 3 days. It is then diluted with water, made alkaline with sodium hydrogen carbonate, and extracted with ethyl acetate. The combined organic extracts are dried and concentrated by evaporation. The residue is purified by chromatography (silica gel; petroleum ether/ethyl acetate=85:15 to 75:25). Yield: 2.1 g (87% of theory); R_{f} value: 0.35 (silica gel; petroleum ether/ethyl acetate=9:1); C₁₉H₂₄N₂O₂ (312.42); mass spectrum: (M−H)⁻=311.

### b. 2,5-dimethyl-4-[N-(2-ethoxycarbonylethylcarbonyl)-N-isopropylamino]-N-benzyl-oxycarbonylaniline

A mixture of 0.9 mL (6.1 mmol) of monoethyl succinate monochloride, 2.1 g (6.7 mmol) of 2,5-dimethyl-4-isopropylaminobenzyloxycarbonylaniline, 3.2 mL (18.3 mmol) of Hünig base, and 74.7 mg (0.6 mmol) of 2-dimethylaminopyridine are stirred in 50 mL of tetrahydrofuran for 5 hours. Then the solvent is distilled off and the residue is purified by chromatography (silica gel; dichloromethane/ethanol=98:2). Yield: 1.9 g (70% of theory); R_{f} value: 0.18 (silica gel; petroleum ether/ethyl acetate=3:1).

### c. 2,5-dimethyl-4-[N-(2-ethoxycarbonylpropionyl)-N-isopropylamino]aniline

Prepared analogously to Example 1b from 2,5-dimethyl-4-[N-(2-ethoxycarbonylethylcarbonyl)-N-isopropylamino]-N-benzyloxycarbonylaniline, 10% palladium on charcoal, and hydrogen in methanol. Yield: 100% of theory; R_{f} value: 0.23 (silica gel; dichloromethane/ethanol=95:5); C₁₇H₂₆N₂O₃ (306.41); mass spectrum: (M+Na)⁺=329.

### d. 4-{N-[2,5-dimethyl-4-(N′-isopropyl-N′-(2-ethoxycarbonylethylcarbonyl)amino)phenylaminocarbonylmethyl]amino}benzonitrile

A mixture of 1.3 g (4.3 mmol) of 2,5-dimethyl-4-[N-(2-ethoxycarbonylpropionyl)-N-isopropylamino]aniline, 1.0 g (5.5 mmol) of N-(4-cyanophenyl)glycine, 1.8 g (5.5 mmol) of O-(benzotriazol-1-yl)-N,N,N′,N′-tetramethyluronium tetrafluoroborate, and 0.8 mL (5.5 mmol) of triethylamine are stirred in 35 mL of dimethylformamide for 4 hours. The reaction mixture is poured onto water and extracted with ethyl acetate. The combined organic extracts are concentrated by evaporation and then purified by chromatography (silica gel; dichloromethane/ethanol=97:3). Yield: 1.7 g (88% of theory); R_{f} value: 0.23 (silica gel; dichloromethane/ethanol=95:5); C₂₆H₃₂N₄O₄ (464.57); mass spectrum: (M+H)⁺=465 and (M+Na)⁺=487

### e. 4-{N-[2,5-dimethyl-4-(N′-isopropyl-N′-(2-ethoxycarbonylethylcarbonyl)amino)phenylaminocarbonylmethyl]amino}benzamidine

Prepared analogously to Example 1d from 4-{N-[2,5-dimethyl-4-(N′-isopropyl-N′-(2-ethoxy-carbonylethylcarbonyl)amino)phenylaminocarbonylmethyl]amino}benzonitrile, ethanol saturated with hydrogen chloride gas, and ammonium acetate. Yield: 79% of theory; C₂₆H₃₅N₅O₄×HCl (481.59/518.06); mass spectrum: (M+H)⁺=482.

### EXAMPLE 47

### 4-{N-[2,5-dimethyl-4-(N′-(2-hydroxycarbonylethylcarbonyl-N′-isopropylamino)phenyl]aminocarbonylmethylamino}benzamidine

0.3 g (0.6 mmol) of 4-{N-[2,5-dimethyl-4-(N′-ethoxycarbonylethylcarbonyl-N′-isopropylamino)phenyl]aminocarbonylmethylamino}benzamidine are stirred in 35 mL of 6 molar hydrochloric acid for 7 hours. Then the mixture is concentrated by evaporation, the residue is taken up in acetone, and again concentrated by evaporation. Yield: 0.3 g (88% of theory); C₂₄H₃₁N₅O₄×HCl (453.55/490.01); mass spectrum: (M+H)⁺=454 (M−H)⁻=452, (M−H+HCl)⁻=488/90 (chlorine isotope).

### EXAMPLE 48

### 4-{N-[2,5-dimethyl-4-(N′-ethoxycarbonylmethylaminocarbonyl-N′-isopropylamino)phenyl]aminocarbonylmethylamino}benzamidine

Prepared analogously to Example 1d from 4-{N-[2,5-dimethyl-4-(N′-ethoxycarbonylmethyl-aminocarbonyl-N′-isopropylamino)phenyl]aminocarbonylmethylamino}benzonitrile, ethanol saturated with hydrogen chloride gas, and ammonium acetate. Yield: 42% of theory; C₂₅H₃₄N₆O₄×HCl (482.59/519.06); mass spectrum: (M+H)⁺=483, (M−H)⁻=481, and (M−H+HCl)⁻=517/519 (chlorine isotope).

### EXAMPLE 49

### 4-{N-[2,5-dimethyl-4-(N′-hydroxycarbonylmethylaminocarbonyl-N′-isopropylamino)phenyl]aminocarbonylmethylamino}benzamidine

Prepared analogously to Example 21 from 4-{N-[2,5-dimethyl-4-(N′-ethoxycarbonylmethyl-aminocarbonyl-N′-isopropylamino)phenyl]aminocarbonylmethylamino}benzamidine, sodium hydroxide in ethanol/water, and subsequent treatment with hydrochloric acid. Yield: 48% of theory; C₂₃H₃₀N₆O₄ (454.53); mass spectrum: (M−H)⁻=453, (M+H)⁺=455, and (M+Na)⁺=477.

### EXAMPLE 50

### 4-{N-[2,5-dimethyl-4-(N′-(3-amino-3-ethoxycarbonylpropionyl)-N′-isopropylamino)phenyl]aminocarbonylmethylamino}benzamidine

Prepared analogously to Example 1d from 4-{N-[2,5-dimethyl-4-(N′-(3-amino-3-ethoxy-carbonylpropionyl)-N′-isopropylamino)phenyl]aminocarbonylmethylamino}benzonitrile, ethanol saturated with hydrogen chloride gas, and ammonium acetate. Yield: 17% of theory; C₂₆H₃₆N₆O₄×2HCl (496.62/569.54); mass spectrum: (M+H)⁺=497.

### EXAMPLE 51

### 4-{N-[2,5-dimethyl-4-(N′-(3-amino-3-hydroxycarbonylpropionyl)-N′-isopropylamino)phenyl]aminocarbonylmethylamino}benzamidine

Prepared analogously to Example 21 from 4-{N-[2,5-dimethyl-4-(N′-(3-amino-3-ethoxy-carbonylpropionyl)-N′-isopropylamino)phenyl]aminocarbonylmethylamino}benzamidine, sodium hydroxide in methanol/water, and subsequent treatment with hydrochloric acid. Yield: 25% of theory; C₂₄H₃₂N₆O₄×2 HCl (468.56/541.48); mass spectrum: (M+H)⁺=469.

### EXAMPLE 52

### 4-{N-[2,5-dimethyl-4-(N′-hydroxycarbonylmethylaminocarbonyl-N′-isopropylamino)phenyl]aminocarbonylmethyl}benzamidine

Prepared analogously to Example 47 from 4-{N-[2,5-dimethyl-4-(N′-ethoxycarbonylmethyl-aminocarbonyl-N′-isopropylamino)phenyl]aminocarbonylmethyl}benzamidine and 6N hydrochloric acid. Yield: 95% of theory; C₂₃H₂₉N₅O₄×HCl (439.52/475.98); mass spectrum: (M+H)⁺=440 and (M−H)⁻=438.

### EXAMPLE 53

### 4-{2-{N-[2,5-dimethyl-4-(N′-hydroxycarbonylmethylaminocarbonyl-N′-isopropylamino)phenyl]aminocarbonyl}ethyl}benzamidine

Prepared analogously to Example 47 from 4-{2-{N-[2,5-dimethyl-4-(N′-ethoxycarbonylmethyl-aminocarbonyl-N′-isopropylamino)phenyl]aminocarbonyl}ethyl}benzamidine and 6N hydrochloric acid. Yield: 91 of theory; C₂₄H₃₁N₅O₄×HCl (453.55/490.01); mass spectrum: (M+H)⁺=454 and (M−H)⁻=452.

### EXAMPLE 54

### Dry Ampoule Containing 75 mg of Active Substance per 10 mL

Composition:

| | | |
|---|---|---|
| Active substance | 75.0 | mg |
| Mannitol | 50.0 | mg |
| water for injections | to 10.0 | mL |

Preparation:

Active substance and mannitol are dissolved in water. After packaging, the solution is freeze-dried. To produce the solution ready for use for injections, the product is dissolved in water.

### EXAMPLE 55

### Dry Ampoule Containing 35 mg of Active Substance per 2 mL

Composition:

| | | |
|---|---|---|
| Active substance | 35.0 | mg |
| Mannitol | 100.0 | mg |
| water for injections | to 2.0 | mL |

Preparation:

Active substance and mannitol are dissolved in water. After packaging, the solution is freeze-dried. To produce the solution ready for use for injections, the product is dissolved in water.

### EXAMPLE 56

### Tablet Containing 50 mg of Active Substance

Composition:

| | |
|---|---|
| (1) Active substance | 50.0 mg |
| (2) Lactose | 98.0 mg |
| (3) Maize starch | 50.0 mg |
| (4) Polyvinylpyrrolidone | 15.0 mg |
| (5) Magnesium stearate | 2.0 mg |
| | 215.0 mg |

Preparation:

(1), (2), and (3) are mixed together and granulated with an aqueous solution of (4). (5) is added to the dried granulated material. From this mixture tablets are pressed, biplanar, faceted on both sides, and with a dividing notch on one side. Diameter of the tablets: 9 mm.

### EXAMPLE 57

### Tablet Containing 350 mg of Active Substance

Composition:

| | |
|---|---|
| (1) Active substance | 350.0 mg |
| (2) Lactose | 136.0 mg |
| (3) Maize starch | 80.0 mg |
| (4) Polyvinylpyrrolidone | 30.0 mg |
| (5) Magnesium stearate | 4.0 mg |
| | 600.0 mg |

Preparation:

(1), (2), and (3) are mixed together and granulated with an aqueous solution of (4). (5) is added to the dried granulated material. From this mixture tablets are pressed, biplanar, faceted on both sides, and with a dividing notch on one side. Diameter of the tablets: 12 mm.

### EXAMPLE 58

### Capsules Containing 50 mg of Active Substance

Composition:

| | |
|---|---|
| (1) Active substance | 50.0 mg |
| (2) Dried maize starch | 58.0 mg |
| (3) Powdered lactose | 50.0 mg |
| (4) Magnesium stearate | 2.0 mg |
| | 160.0 mg |

Preparation:

(1) is triturated with (3). This trituration is added to the mixture of (2) and (4) with vigorous mixing. This powder mixture is packed into size 3 hard gelatine capsules in a capsule filling machine.

### EXAMPLE 59

### Capsules Containing 350 mg of Active Substance

Composition:

| | |
|---|---|
| (1) Active substance | 350.0 mg |
| (2) Dried maize starch | 46.0 mg |
| (3) Powdered lactose | 30.0 mg |
| (4) Magnesium stearate | 4.0 mg |
| | 430.0 mg |

Preparation:

(1) is triturated with (3). This trituration is added to the mixture of (2) and (4) with vigorous mixing. This powder mixture is packed into size 0 hard gelatine capsules in a capsule filling machine.

### EXAMPLE 60

### Suppositories Containing 100 mg of Active Substance

1 suppository contains:

| | |
|---|---|
| Active substance | 100.0 mg |
| Polyethyleneglycol (M.W. 1500) | 600.0 mg |
| Polyethyleneglycol (M.W. 6000) | 460.0 mg |
| Polyethylenesorbitan monostearate | 840.0 mg |
| | 2,000.0 mg |

Preparation:

The polyethyleneglycol is melted together with polyethylene sorbitan monostearate. At 40° C. the ground active substance is homogeneously dispersed in the melt. This is cooled to 38° C. and poured into slightly chilled suppository moulds.

Each of the references cited herein is incorporated by reference herein in its entirety.

We claim:

1. A compound of formula (I) 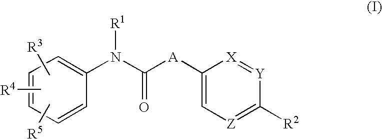
wherein:
A is a methylene group optionally substituted by a C₁₋₃-alkyl group, or
a straight-chain C₂₋₃-alkyl group optionally substituted by a C₁₋₃-alkyl group wherein the methylene group linked to the aromatic group or heteroaromatic group is optionally replaced by an oxygen or sulfur atom or by an —NH— group, wherein the —NH— group is optionally additionally substituted by a C₁₋₃-alkyl, carboxy-C₁₋₃-alkyl, or C₁₋₃-alkoxycarbonyl-C₁₋₃-alkyl group;
R¹ is a hydrogen atom or a C₁₋₃-alkyl group optionally substituted by a carboxy group;
R² is a cyano, aminomethyl, or amidino group optionally substituted by a hydroxy, C₁₋₈-alkoxycarbonyl or benzoyl group;
R³ is a C₁₋₅-alkyl or carboxy-C₁₋₄-alkyl group, each optionally substituted in the alkyl moiety by a C₃₋₇-cycloalkyl, phenyl, pyridyl, pyrrolidino, 2,5-dihydro-1H-pyrrolino, piperidino, or hexamethyleneimino group,
a carbonyl or sulfonyl group which is substituted in each case
by a C₁₋₅-alkyl, C₃₋₇-cycloalkyl, or phenyl group optionally substituted by a C₁₋₃-alkyl or carboxy-C₁₋₃-alkyl group,
by an amino, C₁₋₄-alkylamino, or carboxy-C₁₋₄-alkylamino group substituted by a C₁₋₅-alkyl, C₃₋₇-cycloalkyl, phenyl, phenyl-C₁₋₃-alkyl, pyridyl, or pyridyl-C₁₋₃-alkyl group, or
by a pyrrolidino, 2,5-dihydro-1H-pyrrolino, piperidino, or hexamethyleneimino group optionally substituted by a C₁₋₃-alkyl or carboxy-C₁₋₃-alkyl group,
a carboxy-C₁₋₃-alkylcarbonylamino group optionally substituted in the alkyl moiety by an amino, C₁₋₃-alkylamino, or di-(C₁₋₃-alkyl)-amino group, or an amino, carboxy-C₁₋₃-alkylaminocarbonylamino, carboxy-C₁₋₃-alkylaminocarbonyl-C₁₋₃-alkylcarbonylamino, carboxy-C₁₋₃-alkylaminocarbonyl-C₁₋₃-alkylaminocarbonylamino, amino-C₁₋₃-alkylcarbonylamino, C₁₋₃-alkylamino-C₁₋₃-alkylcarbonylamino, or di-(C₁₋₃-alkyl)-amino-C₁₋₃-alkylcarbonylamino group, wherein in each case in the amino groups thereof the hydrogen atom of the amino group which is linked to the phenyl ring is replaced by a C₁₋₆-alkyl, C₃₋₇-cycloalkyl, phenyl, or pyridyl group, an n-propylene or n-butylene bridge or a phenyl, pyridine, or piperidine ring is optionally fused to the phenyl or pyridyl substituents via two adjacent carbon atoms, or the aromatic substituents are optionally additionally substituted by a C₁₋₃-alkyl, C₁₋₃-alkyloxy, trifluoromethyl, or carboxy group or by 2 to 4 methyl groups,
an amino, carboxy-C₁₋₄-alkylamino, carboxy-C₁₋₃-alkylaminocarbonyl-C₁₋₃-alkylamino, aminocarbonyl-C₁₋₃-alkylamino, C₁₋₃-alkylaminocarbonyl-C₁₋₃-alkylamino, di-(C₁₋₃-alkyl)-aminocarbonyl-C₁₋₃-alkylamino, amino-C₂₋₃-alkylamino, C₁₋₄-alkylamino-C₂₋₃-alkylamino, di-(C₁₋₄-alkyl)-amino-C₂₋₃-alkylamino, pyrrolidinocarbonyl-C₁₋₃-alkylamino, piperidinocarbonyl-C₁₋₃-alkylamino, hexahydroazepinocarbonyl-C₁₋₃-alkylamino, morpholinocarbonyl-C₁₋₃-alkylamino, piperazinocarbonyl-C₁₋₃-alkylamino, or N-(C₁₋₃-alkyl)-piperazinocarbonyl-C₁₋₃-alkylamino group, wherein in each case in the amino groups thereof the hydrogen atom of the amino group which is linked to the phenyl ring is replaced by a C₁₋₅-alkylcarbonyl, C₁₋₅-alkylsulfonyl, C₃₋₇-cycloalkylcarbonyl, C₃₋₇-cycloalkylsulfonyl, benzoyl, phenylsulfonyl, phenyl-C₁₋₃-alkylcarbonyl, phenyl-C₁₋₃-alkylsulfonyl, or pyridinoyl group, an n-propylene or n-butylene bridge or a phenyl, pyridine, or piperidine ring is optionally fused to the phenyl or pyridyl substituents via two adjacent carbon atoms or the aromatic substituents are optionally additionally substituted by a C₁₋₃-alkyl, C₁₋₃-alkyloxy, trifluoromethyl, or carboxy group or by 2 to 4 methyl groups, or
a phenyl, pyridyl, imidazolyl, or pyrazolyl group optionally substituted by one, two, or three C₁₋₃-alkyl groups, wherein in each case the alkyl substituents are identical or different and one of the alkyl substituents is optionally additionally substituted by a carboxy, hydroxysulfonyl, aminosulfonyl, C₁₋₄-alkylaminosulfonyl, di-(C₁₋₄-alkyl)-aminosulfonyl, or C₁₋₄-alkylsulfonyl group;
R⁴ is a fluorine, chlorine, bromine, or iodine atom, or a carboxy, C₁₋₃-alkyl, carboxy-C₁₋₃-alkyl, trifluoromethyl, or C₁₋₃-alkoxy group, or also a hydrogen atom, if
R³ is a C₁₋₅-alkyl or carboxy-C₁₋₄-alkyl group, each substituted in the alkyl moiety by a C₃₋₇-cycloalkyl, phenyl, pyridyl, pyrrolidino, 2,5-dihydro-1H-pyrrolino, piperidino, or hexamethyleneimino group,
an amino, carboxy-C₁₋₄-alkylamino, or carboxy-C₁₋₃-alkylaminocarbonyl-C₁₋₃-alkylamino group, wherein in each case in the amino groups thereof the hydrogen atom of the amino group which is linked to the phenyl ring is replaced by a C₁₋₅-alkylcarbonyl, C₁₋₅-alkylsulfonyl, C₃₋₇-cycloalkylcarbonyl, C₃₋₇-cycloalkylsulfonyl, benzoyl, phenylsulfonyl, phenyl-C₁₋₃-alkylcarbonyl, phenyl-C₁₋₃-alkylsulfonyl, or pyridinoyl group, an n-propylene or n-butylene bridge, a phenyl, pyridine, or piperidine ring is optionally fused to the phenyl or pyridyl substituents via two adjacent carbon atoms or the aromatic substituents are optionally additionally substituted by a C₁₋₃-alkyl, C₁₋₃-alkyloxy, trifluoromethyl, or carboxy group or by 2 to 4 methyl groups; and
R⁵ is a hydrogen, fluorine, chlorine, bromine, or iodine atom, or a C₁₋₃-alkyl or trifluoromethyl group, or
R⁴ and R⁵ together are an n-C₃₋₄-alkylene group,
with the proviso that at least one-of the groups R¹, R⁴, or R⁵ is not a hydrogen atom, and
X, Y, and Z in each case are nitrogen atoms or —CH— groups, with the proviso that at least one of the groups X, Y, and Z is a —CH— group,
wherein, unless otherwise stated:
(i) the hydrogen atoms in the methyl and methoxy groups thereof are optionally wholly or partially replaced by fluorine atoms,
(ii) the carboxy groups are optionally replaced by a group which is converted in vivo into a carboxy group or by a group which is negatively charged under physiological conditions,
(iii) the amino and imino groups are optionally substituted by a group which is cleaved in vivo, and
the prodrugs, tautomers, and salts thereof.

2. The compound of formula (I) according to claim 1, wherein:
A is a methylene group, or
a C₂₋₃-alkyl group wherein the methylene group linked to the aromatic group or heteroaromatic group is optionally replaced by an —NH— group or by an oxygen atom, while the —NH— group may additionally be substituted by a C₁₋₃-alkyl, carboxy-C₁₋₃-alkyl or C₁₋₃-alkoxycarbonyl-C₁₋₃-alkyl group;
R³ is a C₁₋₅-alkyl or carboxy-C₁₋₄-alkyl group, each optionally substituted in the alkyl moiety by a C₃₋₇-cycloalkyl, phenyl, pyridyl, pyrrolidino, 2,5-dihydro-1H-pyrrolino, piperidino, or hexamethyleneimino group,
a carbonyl or sulfonyl group which is substituted in each case
by a C₁₋₅-alkyl or C₃₋₇-cycloalkyl group optionally substituted by a C₁₋₃-alkyl or carboxy-C₁₋₃-alkyl group,
by an amino, C₁₋₄-alkylamino, or carboxy-C₁₋₄-alkylamino group substituted by a C₁₋₅-alkyl, C₃₋₇-cycloalkyl, phenyl, benzyl, or pyridyl group, or
by a pyrrolidino, 2,5-dihydro-1H-pyrrolino, piperidino, or hexamethyleneimino group optionally substituted by a C₁₋₃-alkyl or carboxy-C₁₋₃-alkyl group,
a carboxy-C₁₋₃-alkylcarbonylamino group optionally substituted in the alkyl moiety by an amino group, or an amino, carboxy-C₁₋₃-alkylaminocarbonylamino, carboxy-C₁₋₃-alkylaminocarbonyl-C₁₋₃-alkylcarbonylamino, carboxy-C₁₋₃-alkylaminocarbonyl-C₁₋₃-alkylaminocarbonylamino, or amino-C₁₋₃-alkylcarbonylamino group, wherein in each case in the amino groups thereof the hydrogen atom of the amino group which is linked to the phenyl ring is replaced by a C₁₋₆-alkyl, C₃₋₇-cycloalkyl, phenyl, or pyridyl group, or a phenyl, pyridine, or piperidine ring is optionally fused to the phenyl or pyridyl substituents via two adjacent carbon atoms, or the aromatic substituents are optionally additionally substituted by a C₁₋₃-alkyl, C₁₋₃-alkyloxy, trifluoromethyl, or carboxy group or by 2 to 4 methyl groups,
an amino, carboxy-C₁₋₄-alkylamino, carboxy-C₁₋₃-alkylaminocarbonyl-C₁₋₃-alkylamino, aminocarbonyl-C₁₋₃-alkylamino, pyrrolidinocarbonyl-C₁₋₃-alkylamino, piperidinocarbonyl-C₁₋₃-alkylamino or morpholinocarbonyl-C₁₋₃-alkylamino group, wherein in each case in the amino groups thereof the hydrogen atom of the amino group which is linked to the phenyl ring is replaced by a C₁₋₅-alkylcarbonyl, C₁₋₅-alkylsulfonyl, C₃₋₇-cycloalkylcarbonyl, C₃₋₇-cycloalkylsulfonyl, benzoyl, phenylsulfonyl, phenyl-C₁₋₃-alkylcarbonyl, an n-propylene or n-butylene bridge or a phenyl, pyridine, or piperidine ring is optionally fused to the phenyl or pyridyl substituents via two adjacent carbon atoms or the aromatic substituents are optionally additionally substituted by a C₁₋₃-alkyl, C₁₋₃-alkyloxy, trifluoromethyl, or carboxy group or by 2 to 4 methyl groups,
a phenyl, pyridyl, imidazolyl, or pyrazolyl group optionally substituted by one, two, or three C₁₋₃-alkyl groups, wherein in each case the alkyl substituents are identical or different and one of the alkyl substituents is optionally additionally substituted by a carboxy, hydroxysulfonyl, aminosulfonyl, C₁₋₄-alkylaminosulfonyl, di-(C₁₋₄-alkyl)-aminosulfonyl, or C₁₋₄-alkylsulfonyl group,
R⁴ is a chlorine or bromine atom, a carboxy, C₁₋₃-alkyl, carboxy-C₁₋₃-alkyl, or trifluoromethyl group, or also a hydrogen atom, if
R³ is a C₁₋₅-alkyl or carboxy-C₁₋₄-alkyl group, which is substituted in each case in the alkyl moiety by a C₃₋₇-cycloalkyl, phenyl, pyridyl, pyrrolidino, piperidino, or hexamethyleneimino group,
an amino, carboxy-C₁₋₄-alkylamino, or carboxy-C₁₋₃-alkylaminocarbonyl-C₁₋₃-alkylamino group, wherein in each case in the amino groups thereof the hydrogen atom of the amino group which is linked to the phenyl ring is replaced by a C₁₋₅-alkylcarbonyl, C₁₋₅-alkylsulfonyl, C₃₋₇-cycloalkylcarbonyl, C₃₋₇-cycloalkylsulfonyl, benzoyl, phenylsulfonyl, phenyl-C₁₋₃-alkylcarbonyl, or pyridinoyl group, an n-propylene or n-butylene bridge, or a phenyl, pyridine, or piperidine ring is optionally fused to the phenyl or pyridyl substituents via two adjacent carbon atoms or the aromatic substituents are optionally additionally substituted by a C₁₋₃-alkyl, C₁₋₃-alkyloxy, or trifluoromethyl group or by 2 to 4 methyl groups; and
R⁵ is a hydrogen, chlorine, or bromine atom, or a C₁₋₃-alkyl or trifluoromethyl group or
R⁴ and R⁵ together are an n-C₃₋₄-alkylene group, and
the C₁₋₃-alkyl and benzyl esters, prodrugs, tautomers, and salts thereof.

3. A compound of formula (Ia) 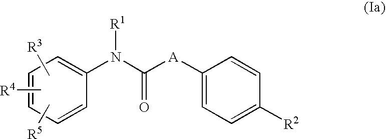
wherein:
A is a methylene group, or
an ethylene group wherein the methylene group linked to the aromatic group is optionally replaced by an oxygen atom or by an —NH— group, wherein the —NH— group is optionally additionally substituted by a methyl, carboxymethyl, or C₁₋₃-alkoxycarbonylmethyl group;
R¹ is a hydrogen atom, or a methyl or ethyl group;
R² is a cyano or aminomethyl group or an amidino group optionally substituted by a hydroxy, C₁₋₈-alkyloxycarbonyl, or benzoyl group;
R³ is a straight-chain or branched C₁₋₅-alkyl group optionally substituted by a phenyl, pyridyl, or piperidino group,
a carbonyl or sulfonyl group which is substituted in each case by a straight-chain or branched C₁₋₅-alkyl, C₃₋₅-cycloalkyl, phenylamino, N-(C₁₋₄-alkyl)phenylamino, N,N-di-(C₁₋₄-alkyl)-amino, N-(C₁₋₄-alkyl)-benzylamino, N-(C₁₋₄-alkyl)-pyridylamino, pyrrolidino, or methylpyrrolidino group,
an amino, methylamino, carboxymethylamino, C₁₋₃-alkoxycarbonylmethylamino, or morpholinocarbonylmethylamino group which is substituted in each case at the amino nitrogen atom by a phenylsulfonyl group optionally substituted by one to four methyl groups, by a phenylsulfonyl group substituted by a trifluoromethyl, carboxy, or C₁₋₃-alkoxycarbonyl group, or by a benzoyl, benzylsulfonyl, naphthylsulfonyl, quinolylsulfonyl, or 1,2,3,4-tetrahydroquinolylsulfonyl group,
a straight-chain or branched C₁₋₅-alkylamino or C₃₋₅-cycloalkylamino group which is substituted in each case at the amino nitrogen atom by a C₂₋₃-alkanoyl group substituted by a carboxy or C₁₋₃-alkoxycarbonyl and/or an amino group, or by a carboxymethylaminocarbonyl or C₁₋₃-alkoxycarbonylmethylaminocarbonyl group, or
a pyrazol-1-yl group substituted by two straight-chain or branched C₁₋₃-alkyl groups;
R⁴ is a chlorine or bromine atom, or a methyl, trifluoromethyl, carboxymethyl, or C₁₋₃-alkoxycarbonylmethyl group or also a hydrogen atom, if
R¹ is an ethyl group, or
R³ is a pyrrolidinocarbonyl group, a carboxymethylamino, or C₁₋₃-alkoxycarbonylmethylamino group wherein in each case the amino nitrogen atom is substituted by a benzoyl group; and
R⁵ is a hydrogen, chlorine, or bromine atom or a methyl group, or
R⁴ and R⁵ together are an n-propylene group,
with the proviso that at least two of the groups R¹, R⁴, and R⁵ are not hydrogen atoms,
wherein, unless otherwise stated the hydrogen atoms in the methyl and methoxy groups thereof are optionally wholly or partially replaced by fluorine atoms, and
the prodrugs, tautomers, and salts thereof.

4. The compound of formula (Ia) according to claim 3, wherein:
R⁴ is a chlorine or bromine atom, or a methyl or trifluoromethyl group,
and the prodrugs, tautomers, and salts thereof

5. The compound of formula (Ia) according to claim 3, wherein:
A is an ethylene group wherein the methylene group linked to the aromatic group is optionally replaced by an —NH— group;
R¹ is a hydrogen atom, or a methyl or ethyl group;
R² is an amidino group;
R³ is a C₃₋₅-alkyl group,
a carbonyl group which is substituted by a straight-chain or branched C₁₋₅-alkyl, C₃₋₅-cycloalkyl, N,N-di-(C₁₋₄-alkyl)-amino, N-(C₁₋₄-alkyl)-benzylamino, N-(C₁₋₄-alkyl)-pyridylamino, pyrrolidino, or 2-methylpyrrolidino group,
a straight-chain or branched C₁₋₅-alkylamino or C₃₋₅-cycloalkylamino group which is substituted in each case at the amino nitrogen atom by a C₂₋₃-alkanoyl group substituted by a carboxy, or C₁₋₃-alkoxycarbonyl and/or an amino group, or by a carboxymethylaminocarbonyl or C₁₋₃-alkoxycarbonylmethylaminocarbonyl group, or
a pyrazol-1-yl group substituted by two straight-chain or branched C₁₋₃-alkyl groups;
R⁴ is a chlorine or bromine atom, or a methyl or trifluoromethyl group; and
R⁵ is a hydrogen, chlorine, or bromine atom or a methyl group,
with the proviso that at least one of the groups R¹ or R⁵ is not a hydrogen atom,
and the prodrugs, tautomers, and salts thereof.

6. The compound of formula (Ia) according to claim 5, wherein R³ is in the 4 position, and the prodrugs, tautomers, and salts thereof.

7. A compound selected from:
(a) 4-{N-[2,5-dimethyl-4-(2-methylpyrrolidinocarbonyl)phenylaminocarbonylmethyl]-amino}benzamidine,
(b) 4-[N-(2,5-dimethyl-4-isopropylcarbonylphenylaminocarbonylmethyl)amino]benzamidine; and
(c) 4-{N-[2,5-dimethyl-4-(N′-isopropyl-N′-(2-ethoxycarbonylethylcarbonyl)amino)phenylaminocarbonylmethyl]amino}benzamidine,
and the prodrugs, tautomers, and salts thereof.

8. A compound selected from:
(a) 4-{N-[2,5-dimethyl-4-(2-methylpyrrolidinocarbonyl)phenylaminocarbonylmethyl]-amino}benzamidine,
(b) 4-[N-(2,5-dimethyl-4-isopropylcarbonylphenylaminocarbonylmethyl)amino]benzamidine; and
(c) 4-{N-[2,5-dimethyl-4-(N′-isopropyl-N′-(2-ethoxycarbonylethylcarbonyl)amino)phenylaminocarbonylmethyl]amino}benzamidine,
and the tautomers and salts thereof.

9. The compound according to one of claims 1 to 8, wherein R² is an amidino group, and the tautomers and physiologically acceptable salts thereof.

10. A pharmaceutical compositions comprising a compound according to claim 9 and one or more inert carriers and/or diluents.
